SEQUENCE LISTING (326 sequences)

SEQ ID NO: 1 - DNA, 22 bp, synthetic construct
  source 1..22: mol_type = other DNA
ttctccccaaatcacagcaccc

SEQ ID NO: 2 - DNA, 23 bp, synthetic construct
  source 1..23: mol_type = other DNA
cgtaaggatgggcaaaacgttca

SEQ ID NO: 3 - DNA, 28 bp, synthetic construct
  source 1..28: mol_type = other DNA
aagcagtttgatgtgagctctaaaacga

SEQ ID NO: 4 - DNA, 28 bp, synthetic construct
  source 1..28: mol_type = other DNA
gccagtagaaattctaaaacaaaaccca

SEQ ID NO: 5 - DNA, 23 bp, synthetic construct
  source 1..23: mol_type = other DNA
atcattttctcagtgcgcaacac

SEQ ID NO: 6 - DNA, 28 bp, synthetic construct
  source 1..28: mol_type = other DNA
ctttgatgtcagattttcttaggaccga

SEQ ID NO: 7 - DNA, 24 bp, synthetic construct
  source 1..24: mol_type = other DNA
cactcactttgtgaccattccggt

SEQ ID NO: 8 - DNA, 26 bp, synthetic construct
  source 1..26: mol_type = other DNA
ctgaaggactactacctcttctacct

SEQ ID NO: 9 - DNA, 22 bp, synthetic construct
  source 1..22: mol_type = other DNA
gatgagcacctcgagaagacct

SEQ ID NO: 10 - DNA, 22 bp, synthetic construct
  source 1..22: mol_type = other DNA
gatggctggtaccgatcatctc

SEQ ID NO: 11 - DNA, 24 bp, synthetic construct
  source 1..24: mol_type = other DNA
tagcaacgccaatctcagagaggt

SEQ ID NO: 12 - DNA, 28 bp, synthetic construct
  source 1..28: mol_type = other DNA
tgctgtaagcactctacacatatcaatt

SEQ ID NO: 13 - DNA, 25 bp, synthetic construct
  source 1..25: mol_type = other DNA
ataagctatgctgagggaagtctgg

SEQ ID NO: 14 - DNA, 22 bp, synthetic construct
  source 1..22: mol_type = other DNA
atgaagctggctcagtcaactc

SEQ ID NO: 15 - DNA, 26 bp, synthetic construct
  source 1..26: mol_type = other DNA
catagtttccagagtggtttgcaggc

SEQ ID NO: 16 - DNA, 23 bp, synthetic construct
  source 1..23: mol_type = other DNA
atgagatgggtggtggacagtta

SEQ ID NO: 17 - DNA, 23 bp, synthetic construct
  source 1..23: mol_type = other DNA
tcctaggcgtaaatggatgagag

SEQ ID NO: 18 - DNA, 26 bp, synthetic construct
  source 1..26: mol_type = other DNA
atgatagaatgtagaacccagccatc

SEQ ID NO: 19 - DNA, 26 bp, synthetic construct
  source 1..26: mol_type = other DNA
cttaatgataatgctggcagagtctg

SEQ ID NO: 20 - DNA, 28 bp, synthetic construct
  source 1..28: mol_type = other DNA
ttgatatatttccaaacacctgtgtgct

SEQ ID NO: 21 - DNA, 25 bp, synthetic construct
  source 1..25: mol_type = other DNA
atctttaagagtccgctttgtgttt

SEQ ID NO: 22 - DNA, 25 bp, synthetic construct
  source 1..25: mol_type = other DNA
aatgtctccctgaggaattctacct

SEQ ID NO: 23 - DNA, 28 bp, synthetic construct
  source 1..28: mol_type = other DNA
gcaagataccaagttcttgaataaggag

SEQ ID NO: 24 - DNA, 24 bp, synthetic construct
  source 1..24: mol_type = other DNA
cacctcctccataatccacaagtg

SEQ ID NO: 25 - DNA, 26 bp, synthetic construct
  source 1..26: mol_type = other DNA
cacaaaatgagaggaaggttactgag

SEQ ID NO: 26 - DNA, 24 bp, synthetic construct
  source 1..24: mol_type = other DNA
caaaggaggtcacatcaccatctc

SEQ ID NO: 27 - DNA, 28 bp, synthetic construct
  source 1..28: mol_type = other DNA
gaatctacttcacttgaatgcatgttaa

SEQ ID NO: 28 - DNA, 27 bp, synthetic construct
  source 1..27: mol_type = other DNA
ggagaaactaagccatatatccatggt

SEQ ID NO: 29 - DNA, 27 bp, synthetic construct
  source 1..27: mol_type = other DNA
tcaatcaccatgttttgactcagttta

SEQ ID NO: 30 - DNA, 27 bp, synthetic construct
  source 1..27: mol_type = other DNA
aattccctcagagagattattcgatgc

SEQ ID NO: 31 - DNA, 27 bp, synthetic construct
  source 1..27: mol_type = other DNA
gattgttctatgatgctgggtaggggg

SEQ ID NO: 32 - DNA, 25 bp, synthetic construct
  source 1..25: mol_type = other DNA
tgtgttcaggataccatgccattag

SEQ ID NO: 33 - DNA, 23 bp, synthetic construct
  source 1..23: mol_type = other DNA
aggaaggctaatgacctcgcaat

SEQ ID NO: 34 - DNA, 27 bp, synthetic construct
  source 1..27: mol_type = other DNA
gacaccaccagaacttcttgcttatta

SEQ ID NO: 35 - DNA, 27 bp, synthetic construct
  source 1..27: mol_type = other DNA
tctcacttatgatgctgctagactgac

SEQ ID NO: 36 - DNA, 23 bp, synthetic construct
  source 1..23: mol_type = other DNA
aagagcacatgagatccgcaatc

SEQ ID NO: 37 - DNA, 24 bp, synthetic construct
  source 1..24: mol_type = other DNA
ggagcttgctagagaagatcacgg

SEQ ID NO: 38 - DNA, 25 bp, synthetic construct
  source 1..25: mol_type = other DNA
attgctcagccacaaaagattctca

SEQ ID NO: 39 - DNA, 29 bp, synthetic construct
  source 1..29: mol_type = other DNA
tttaatagtgaaagcagcaaggttcttca

SEQ ID NO: 40 - DNA, 28 bp, synthetic construct
  source 1..28: mol_type = other DNA
ttttcccagataaatttcagtgtcagct

SEQ ID NO: 41 - DNA, 21 bp, synthetic construct
  source 1..21: mol_type = other DNA
aaagagttgcatgccgtctgt

SEQ ID NO: 42 - DNA, 27 bp, synthetic construct
  source 1..27: mol_type = other DNA
gttctaggtgtcgtttgccttaagtta

SEQ ID NO: 43 - DNA, 27 bp, synthetic construct
  source 1..27: mol_type = other DNA
agttctcaaagacttcaagacaagtta

SEQ ID NO: 44 - DNA, 28 bp, synthetic construct
  source 1..28: mol_type = other DNA
tcttttctccacttttcagacttcttgt

SEQ ID NO: 45 - DNA, 28 bp, synthetic construct
  source 1..28: mol_type = other DNA
accatctcagtgctgaaagaaatataaa

SEQ ID NO: 46 - DNA, 25 bp, synthetic construct
  source 1..25: mol_type = other DNA
ccagactcatcacgtcatccagata

SEQ ID NO: 47 - DNA, 25 bp, synthetic construct
  source 1..25: mol_type = other DNA
gtgacagctaggtttcattactgcg

SEQ ID NO: 48 - DNA, 26 bp, synthetic construct
  source 1..26: mol_type = other DNA
aagccaggatttacccatttatggag

SEQ ID NO: 49 - DNA, 25 bp, synthetic construct
  source 1..25: mol_type = other DNA
ccaagctccctgtgatatttctaaa

SEQ ID NO: 50 - DNA, 27 bp, synthetic construct
  source 1..27: mol_type = other DNA
actggaagagtcttttgtttcatagcc

SEQ ID NO: 51 - DNA, 26 bp, synthetic construct
  source 1..26: mol_type = other DNA
gccttttccccttattctttaaacaa

SEQ ID NO: 52 - DNA, 28 bp, synthetic construct
  source 1..28: mol_type = other DNA
tgttatctcaccactcacacattaactt

SEQ ID NO: 53 - DNA, 23 bp, synthetic construct
  source 1..23: mol_type = other DNA
ctcagtagtgaactgcctcaggg

SEQ ID NO: 54 - DNA, 28 bp, synthetic construct
  source 1..28: mol_type = other DNA
ccttccccaacactctctaaatatttgc

SEQ ID NO: 55 - DNA, 25 bp, synthetic construct
  source 1..25: mol_type = other DNA
atgcaacactgcacctgagaatatg

SEQ ID NO: 56 - DNA, 26 bp, synthetic construct
  source 1..26: mol_type = other DNA
taccccctaaaaggttttgaatgcag

SEQ ID NO: 57 - DNA, 26 bp, synthetic construct
  source 1..26: mol_type = other DNA
aaccaactaatctactgaaggactgg

SEQ ID NO: 58 - DNA, 24 bp, synthetic construct
  source 1..24: mol_type = other DNA
caagagggacaccatatgtcaagg

SEQ ID NO: 59 - DNA, 26 bp, synthetic construct
  source 1..26: mol_type = other DNA
catttttgaaggctcccatattgcat

SEQ ID NO: 60 - DNA, 26 bp, synthetic construct
  source 1..26: mol_type = other DNA
aaatgtgcagaaagattccaaaggag

SEQ ID NO: 61 - DNA, 23 bp, synthetic construct
  source 1..23: mol_type = other DNA
aggtacccatcaacctctttgtt

SEQ ID NO: 62 - DNA, 25 bp, synthetic construct
  source 1..25: mol_type = other DNA
acctatgtggctgtacaatttgtcc

SEQ ID NO: 63 - DNA, 26 bp, synthetic construct
  source 1..26: mol_type = other DNA
gaagtctactcacctccaggtatacc

SEQ ID NO: 64 - DNA, 26 bp, synthetic construct
  source 1..26: mol_type = other DNA
ccaagccccaaagagtctgattttat

SEQ ID NO: 65 - DNA, 26 bp, synthetic construct
  source 1..26: mol_type = other DNA
aagagctgaaacaagagcattgtgca

SEQ ID NO: 66 - DNA, 27 bp, synthetic construct
  source 1..27: mol_type = other DNA
ccacattgtaactcctagaccaagaag

SEQ ID NO: 67 - DNA, 27 bp, synthetic construct
  source 1..27: mol_type = other DNA
acacagagtttaaggttccttccagaa

SEQ ID NO: 68 - DNA, 26 bp, synthetic construct
  source 1..26: mol_type = other DNA
gggtcacttcaggataataagctcct

SEQ ID NO: 69 - DNA, 26 bp, synthetic construct
  source 1..26: mol_type = other DNA
gatttgtgtcttctgcattcacagct

SEQ ID NO: 70 - DNA, 22 bp, synthetic construct
  source 1..22: mol_type = other DNA
ggctgctcttgtacagcatctc

SEQ ID NO: 71 - DNA, 26 bp, synthetic construct
  source 1..26: mol_type = other DNA
taaatactgtctgcccatgactcctc

SEQ ID NO: 72 - DNA, 27 bp, synthetic construct
  source 1..27: mol_type = other DNA
agagctttggttttaatgctattccct

SEQ ID NO: 73 - DNA, 28 bp, synthetic construct
  source 1..28: mol_type = other DNA
tcactatatttttgaggacaccaaccac

SEQ ID NO: 74 - DNA, 27 bp, synthetic construct
  source 1..27: mol_type = other DNA
tgttggtgccaagtacatctataagaa

SEQ ID NO: 75 - DNA, 33 bp, synthetic construct
  source 1..33: mol_type = other DNA
ttttaagaaagaataaagaaggacagaaagcca

SEQ ID NO: 76 - DNA, 32 bp, synthetic construct
  source 1..32: mol_type = other DNA
gatcatgctatcactaagaaaattatggcaaa

SEQ ID NO: 77 - DNA, 23 bp, synthetic construct
  source 1..23: mol_type = other DNA
tgtctaatggccgctgtagtaaa

SEQ ID NO: 78 - DNA, 27 bp, synthetic construct
  source 1..27: mol_type = other DNA
cttggcaatttaagatgctcaggaatt

SEQ ID NO: 79 - DNA, 28 bp, synthetic construct
  source 1..28: mol_type = other DNA
aattctatctcatccatcttgagtgcat

SEQ ID NO: 80 - DNA, 25 bp, synthetic construct
  source 1..25: mol_type = other DNA
tattacagagtgctgcaggtcattc

SEQ ID NO: 81 - DNA, 28 bp, synthetic construct
  source 1..28: mol_type = other DNA
caaagctagtttctaagtaagccattgc

SEQ ID NO: 82 - DNA, 28 bp, synthetic construct
  source 1..28: mol_type = other DNA
tttttgccagagtttttagtgtactcct

SEQ ID NO: 83 - DNA, 28 bp, synthetic construct
  source 1..28: mol_type = other DNA
ataatggttgaagggtagaatacacgca

SEQ ID NO: 84 - DNA, 24 bp, synthetic construct
  source 1..24: mol_type = other DNA
tcgttcagatgagcatgtggttag

SEQ ID NO: 85 - DNA, 23 bp, synthetic construct
  source 1..23: mol_type = other DNA
tacttgtagcagagggccttatc

SEQ ID NO: 86 - DNA, 26 bp, synthetic construct
  source 1..26: mol_type = other DNA
gttagacagaagttaggcatggagtt

SEQ ID NO: 87 - DNA, 28 bp, synthetic construct
  source 1..28: mol_type = other DNA
taatggaagtactgtttcagttctgcaa

SEQ ID NO: 88 - DNA, 28 bp, synthetic construct
  source 1..28: mol_type = other DNA
aaaaatgttttccttttcttcctagggc

SEQ ID NO: 89 - DNA, 26 bp, synthetic construct
  source 1..26: mol_type = other DNA
catttaccaatgttggctaatacaca

SEQ ID NO: 90 - DNA, 24 bp, synthetic construct
  source 1..24: mol_type = other DNA
agaacagcataggaaggcacttag

SEQ ID NO: 91 - DNA, 27 bp, synthetic construct
  source 1..27: mol_type = other DNA
aaattttgggtcttaccatggtttcaa

SEQ ID NO: 92 - DNA, 24 bp, synthetic construct
  source 1..24: mol_type = other DNA
ttgtgtttttaactggaggccctt

SEQ ID NO: 93 - DNA, 27 bp, synthetic construct
  source 1..27: mol_type = other DNA
atatttaaacaaaggctctgggtgtaa

SEQ ID NO: 94 - DNA, 25 bp, synthetic construct
  source 1..25: mol_type = other DNA
cagggacttctctagtttcatgtgt

SEQ ID NO: 95 - DNA, 24 bp, synthetic construct
  source 1..24: mol_type = other DNA
tgggaacaaagtctcggatgtact

SEQ ID NO: 96 - DNA, 28 bp, synthetic construct
  source 1..28: mol_type = other DNA
cagcaggacattgacagatactcattat

SEQ ID NO: 97 - DNA, 25 bp, synthetic construct
  source 1..25: mol_type = other DNA
agaaaaatggcagagaccttgacac

SEQ ID NO: 98 - DNA, 28 bp, synthetic construct
  source 1..28: mol_type = other DNA
tctactttctgttctctttgtgtttccg

SEQ ID NO: 99 - DNA, 25 bp, synthetic construct
  source 1..25: mol_type = other DNA
caatcacattgttccctagtgtctc

SEQ ID NO: 100 - DNA, 26 bp, synthetic construct
  source 1..26: mol_type = other DNA
aggtgacattgacagagttgcaaata

SEQ ID NO: 101 - DNA, 27 bp, synthetic construct
  source 1..27: mol_type = other DNA
aataaatcgcaatggaagcaacaggaa

SEQ ID NO: 102 - DNA, 24 bp, synthetic construct
  source 1..24: mol_type = other DNA
ctcctagggcttgtgagtctcata

SEQ ID NO: 103 - DNA, 24 bp, synthetic construct
  source 1..24: mol_type = other DNA
gttgccaatcttaccacacctcca

SEQ ID NO: 104 - DNA, 26 bp, synthetic construct
  source 1..26: mol_type = other DNA
agcctttttcttaggacctgacatag

SEQ ID NO: 105 - DNA, 27 bp, synthetic construct
  source 1..27: mol_type = other DNA
tgaactgatccaacttctctctcactg

SEQ ID NO: 106 - DNA, 25 bp, synthetic construct
  source 1..25: mol_type = other DNA
ctcagtgccattgcttatcttcctt

SEQ ID NO: 107 - DNA, 28 bp, synthetic construct
  source 1..28: mol_type = other DNA
caacaaaagagaaaatctgtatagccag

SEQ ID NO: 108 - DNA, 28 bp, synthetic construct
  source 1..28: mol_type = other DNA
tttctgcagttgttcatcttctacgtta

SEQ ID NO: 109 - DNA, 28 bp, synthetic construct
  source 1..28: mol_type = other DNA
ttattggtctgctcagagtttacatcag

SEQ ID NO: 110 - DNA, 28 bp, synthetic construct
  source 1..28: mol_type = other DNA
acagaacattctacccaagattctatgc

SEQ ID NO: 111 - DNA, 25 bp, synthetic construct
  source 1..25: mol_type = other DNA
caacatttttcatgtggcccctact

SEQ ID NO: 112 - DNA, 23 bp, synthetic construct
  source 1..23: mol_type = other DNA
ggaacaaaacaaggtgcggtttt

SEQ ID NO: 113 - DNA, 24 bp, synthetic construct
  source 1..24: mol_type = other DNA
agtgttctgccagggtcaaaataa

SEQ ID NO: 114 - DNA, 25 bp, synthetic construct
  source 1..25: mol_type = other DNA
attgaatgccaaaacctcagggata

SEQ ID NO: 115 - DNA, 28 bp, synthetic construct
  source 1..28: mol_type = other DNA
cagacaagctgatctgatatttctttag

SEQ ID NO: 116 - DNA, 25 bp, synthetic construct
  source 1..25: mol_type = other DNA
gccgcctaagggatttaccaatatg

SEQ ID NO: 117 - DNA, 26 bp, synthetic construct
  source 1..26: mol_type = other DNA
aagacctgagtagcttctgttttctc

SEQ ID NO: 118 - DNA, 25 bp, synthetic construct
  source 1..25: mol_type = other DNA
ggtgctgtaattcccctaaaagcaa

SEQ ID NO: 119 - DNA, 24 bp, synthetic construct
  source 1..24: mol_type = other DNA
caaggaataaagcagtgtgtgcct

SEQ ID NO: 120 - DNA, 26 bp, synthetic construct
  source 1..26: mol_type = other DNA
cctcaagaatcctggaaaatgtcagc

SEQ ID NO: 121 - DNA, 33 bp, synthetic construct
  source 1..33: mol_type = other DNA
acactattttaaattagtcaacagttaagcata

SEQ ID NO: 122 - DNA, 29 bp, synthetic construct
  source 1..29: mol_type = other DNA
ctgaaacatcactcaaaataaaaggcatt

SEQ ID NO: 123 - DNA, 26 bp, synthetic construct
  source 1..26: mol_type = other DNA
tctacaactaagccttttaaccgaga

SEQ ID NO: 124 - DNA, 28 bp, synthetic construct
  source 1..28: mol_type = other DNA
atttcacagttctctcttgatcatgtca

SEQ ID NO: 125 - DNA, 28 bp, synthetic construct
  source 1..28: mol_type = other DNA
gaatgcacagagaaattcttagaggtca

SEQ ID NO: 126 - DNA, 26 bp, synthetic construct
  source 1..26: mol_type = other DNA
ctccacctcttgtcttctagaaccat

SEQ ID NO: 127 - DNA, 28 bp, synthetic construct
  source 1..28: mol_type = other DNA
tctttgtattcactattcttgtggctaa

SEQ ID NO: 128 - DNA, 25 bp, synthetic construct
  source 1..25: mol_type = other DNA
tttcaagatgggatggagaaagcta

SEQ ID NO: 129 - DNA, 25 bp, synthetic construct
  source 1..25: mol_type = other DNA
ccctattccaaacctgtacctacct

SEQ ID NO: 130 - DNA, 26 bp, synthetic construct
  source 1..26: mol_type = other DNA
ccccagtcacccacctaacatttaat

SEQ ID NO: 131 - DNA, 21 bp, synthetic construct
  source 1..21: mol_type = other DNA
gagcctgagactctgctacca

SEQ ID NO: 132 - DNA, 19 bp, synthetic construct
  source 1..19: mol_type = other DNA
gggagtcccacgagcactg

SEQ ID NO: 133 - DNA, 28 bp, synthetic construct
  source 1..28: mol_type = other DNA
aagtgtagtctgtgcaacaagtttctta

SEQ ID NO: 134 - DNA, 27 bp, synthetic construct
  source 1..27: mol_type = other DNA
atacaaggatttagagaccacagcatc

SEQ ID NO: 135 - DNA, 28 bp, synthetic construct
  source 1..28: mol_type = other DNA
ctttggagaaaactgatgagtttagctt

SEQ ID NO: 136 - DNA, 25 bp, synthetic construct
  source 1..25: mol_type = other DNA
cctctgtcttcttaactggctgtag

SEQ ID NO: 137 - DNA, 26 bp, synthetic construct
  source 1..26: mol_type = other DNA
taagttggaatcaccaccattgaccc

SEQ ID NO: 138 - DNA, 28 bp, synthetic construct
  source 1..28: mol_type = other DNA
attcataactcctccacacatctcagta

SEQ ID NO: 139 - DNA, 23 bp, synthetic construct
  source 1..23: mol_type = other DNA
tgagcttggagcagtaaagcagg

SEQ ID NO: 140 - DNA, 26 bp, synthetic construct
  source 1..26: mol_type = other DNA
agtgacatcatgttgactctaactcg

SEQ ID NO: 141 - DNA, 28 bp, synthetic construct
  source 1..28: mol_type = other DNA
aacttgttgcagattcatggaatcattt

SEQ ID NO: 142 - DNA, 26 bp, synthetic construct
  source 1..26: mol_type = other DNA
aaagagaataacagtttgaccttggc

SEQ ID NO: 143 - DNA, 28 bp, synthetic construct
  source 1..28: mol_type = other DNA
atgacagaagagatttctctccagtttg

SEQ ID NO: 144 - DNA, 26 bp, synthetic construct
  source 1..26: mol_type = other DNA
actcgattctttccatttccatgtcg

SEQ ID NO: 145 - DNA, 25 bp, synthetic construct
  source 1..25: mol_type = other DNA
ttaaagtctcctgtgtacacggttg

SEQ ID NO: 146 - DNA, 28 bp, synthetic construct
  source 1..28: mol_type = other DNA
ctgtaccaatcaagagaagtaggatgga

SEQ ID NO: 147 - DNA, 30 bp, synthetic construct
  source 1..30: mol_type = other DNA
gatatttgtaaggtattctggcctaaaaaa

SEQ ID NO: 148 - DNA, 31 bp, synthetic construct
  source 1..31: mol_type = other DNA
aagtattgaagtgatagttttacagtttcct

SEQ ID NO: 149 - DNA, 26 bp, synthetic construct
  source 1..26: mol_type = other DNA
agacttggaatcattcttcacagggt

SEQ ID NO: 150 - DNA, 25 bp, synthetic construct
  source 1..25: mol_type = other DNA
gccagaattagcagttagcagtcat

SEQ ID NO: 151 - DNA, 28 bp, synthetic construct
  source 1..28: mol_type = other DNA
tttcttcctctaagtggcctcataaata

SEQ ID NO: 152 - DNA, 28 bp, synthetic construct
  source 1..28: mol_type = other DNA
agaattagtaagctctttcacttgcagt

SEQ ID NO: 153 - DNA, 28 bp, synthetic construct
  source 1..28: mol_type = other DNA
actagaagcattagaccagattacctgc

SEQ ID NO: 154 - DNA, 25 bp, synthetic construct
  source 1..25: mol_type = other DNA
acagtccattagtgatgggtttgtt

SEQ ID NO: 155 - DNA, 25 bp, synthetic construct
  source 1..25: mol_type = other DNA
cagaaaaacagactggtccaaagtc

SEQ ID NO: 156 - DNA, 26 bp, synthetic construct
  source 1..26: mol_type = other DNA
cactggtttgggaatagagtgctaag

SEQ ID NO: 157 - DNA, 28 bp, synthetic construct
  source 1..28: mol_type = other DNA
cccccagaaaagtatgttttaagactct

SEQ ID NO: 158 - DNA, 27 bp, synthetic construct
  source 1..27: mol_type = other DNA
ccaagaccagagagataacaaatgcaa

SEQ ID NO: 159 - DNA, 26 bp, synthetic construct
  source 1..26: mol_type = other DNA
tttctgaatgtggcctacagtttcac

SEQ ID NO: 160 - DNA, 27 bp, synthetic construct
  source 1..27: mol_type = other DNA
atggaattcgaaatgatgaagcaatga

SEQ ID NO: 161 - DNA, 27 bp, synthetic construct
  source 1..27: mol_type = other DNA
tgttgcttatgctgttgttcttcaccc

SEQ ID NO: 162 - DNA, 27 bp, synthetic construct
  source 1..27: mol_type = other DNA
gtttgtacttctttaaagcagggactg

SEQ ID NO: 163 - DNA, 25 bp, synthetic construct
  source 1..25: mol_type = other DNA
ggaggtcaagaagccttagtttctc

SEQ ID NO: 164 - DNA, 25 bp, synthetic construct
  source 1..25: mol_type = other DNA
atcgtggcgcattatctcttacatc

SEQ ID NO: 165 - DNA, 27 bp, synthetic construct
  source 1..27: mol_type = other DNA
gcattctagccattggacaattttgta

SEQ ID NO: 166 - DNA, 28 bp, synthetic construct
  source 1..28: mol_type = other DNA
taggtctgcaataatttccctctactca

SEQ ID NO: 167 - DNA, 26 bp, synthetic construct
  source 1..26: mol_type = other DNA
actgttaaggtcaatgacgcagagta

SEQ ID NO: 168 - DNA, 28 bp, synthetic construct
  source 1..28: mol_type = other DNA
ttactaaaggacttggtaggtgcacata

SEQ ID NO: 169 - DNA, 28 bp, synthetic construct
  source 1..28: mol_type = other DNA
tttggcccatggatagaaataaaatgtt

SEQ ID NO: 170 - DNA, 28 bp, synthetic construct
  source 1..28: mol_type = other DNA
tttgaaaggcttttgttatcaagggcta

SEQ ID NO: 171 - DNA, 24 bp, synthetic construct
  source 1..24: mol_type = other DNA
ccattccctattcagtggactctt

SEQ ID NO: 172 - DNA, 24 bp, synthetic construct
  source 1..24: mol_type = other DNA
agactcagtgaggtcatgactcaa

SEQ ID NO: 173 - DNA, 24 bp, synthetic construct
  source 1..24: mol_type = other DNA
gagcatcttctccaccagtttggc

SEQ ID NO: 174 - DNA, 23 bp, synthetic construct
  source 1..23: mol_type = other DNA
ttgtctcctttcagcacagaacc

SEQ ID NO: 175 - DNA, 27 bp, synthetic construct
  source 1..27: mol_type = other DNA
aaagcccacattttgttaacatgactc

SEQ ID NO: 176 - DNA, 26 bp, synthetic construct
  source 1..26: mol_type = other DNA
atgtaacttctctgaacagggaagag

SEQ ID NO: 177 - DNA, 23 bp, synthetic construct
  source 1..23: mol_type = other DNA
aagcagcgatttccatgttgccc

SEQ ID NO: 178 - DNA, 23 bp, synthetic construct
  source 1..23: mol_type = other DNA
ggctgattgtggagatgtctcct

SEQ ID NO: 179 - DNA, 25 bp, synthetic construct
  source 1..25: mol_type = other DNA
agaagccaaagctccctaatagctc

SEQ ID NO: 180 - DNA, 27 bp, synthetic construct
  source 1..27: mol_type = other DNA
gagccagttttgttaaagacacaatgt

SEQ ID NO: 181 - DNA, 27 bp, synthetic construct
  source 1..27: mol_type = other DNA
ttgctctgaatagtgctttcagtagtg

SEQ ID NO: 182 - DNA, 28 bp, synthetic construct
  source 1..28: mol_type = other DNA
cagcactttctaaatagtgataggcaag

SEQ ID NO: 183 - DNA, 26 bp, synthetic construct
  source 1..26: mol_type = other DNA
cagctgcttatagttttgttaagaag

SEQ ID NO: 184 - DNA, 25 bp, synthetic construct
  source 1..25: mol_type = other DNA
ggacccctagataatgtcaggattg

SEQ ID NO: 185 - DNA, 28 bp, synthetic construct
  source 1..28: mol_type = other DNA
cttttgagatcatggaaaattccagttg

SEQ ID NO: 186 - DNA, 28 bp, synthetic construct
  source 1..28: mol_type = other DNA
cagaaagaggaacttaagaagatgtggt

SEQ ID NO: 187 - DNA, 27 bp, synthetic construct
  source 1..27: mol_type = other DNA
ggagttctaagcaatgagatgctaatt

SEQ ID NO: 188 - DNA, 26 bp, synthetic construct
  source 1..26: mol_type = other DNA
tttcccataattcccaaagcatggta

SEQ ID NO: 189 - DNA, 26 bp, synthetic construct
  source 1..26: mol_type = other DNA
agcatcatttcatgcttttgaagttt

SEQ ID NO: 190 - DNA, 23 bp, synthetic construct
  source 1..23: mol_type = other DNA
accacctcctgtaacaacatccg

SEQ ID NO: 191 - DNA, 27 bp, synthetic construct
  source 1..27: mol_type = other DNA
agaacccatagggaaacaaaggtatgt

SEQ ID NO: 192 - DNA, 28 bp, synthetic construct
  source 1..28: mol_type = other DNA
tttctctcctttcagggaacattacatc

SEQ ID NO: 193 - DNA, 27 bp, synthetic construct
  source 1..27: mol_type = other DNA
cattcagtatctgtgtgcctcaatgat

SEQ ID NO: 194 - DNA, 26 bp, synthetic construct
  source 1..26: mol_type = other DNA
ctgcagggttttctatccagaacaat

SEQ ID NO: 195 - DNA, 26 bp, synthetic construct
  source 1..26: mol_type = other DNA
cagaatgatgagctgtgcagatagcc

SEQ ID NO: 196 - DNA, 24 bp, synthetic construct
  source 1..24: mol_type = other DNA
gctgtctctatgaacatccctacc

SEQ ID NO: 197 - DNA, 24 bp, synthetic construct
  source 1..24: mol_type = other DNA
tgtgatggagtttatggccaacgg

SEQ ID NO: 198 - DNA, 25 bp, synthetic construct
  source 1..25: mol_type = other DNA
ttatgccccaaatttcactgcttag

SEQ ID NO: 199 - DNA, 21 bp, synthetic construct
  source 1..21: mol_type = other DNA
aactccggtctatccaggtcc

SEQ ID NO: 200 - DNA, 22 bp, synthetic construct
  source 1..22: mol_type = other DNA
tgatcccatgggactactcacg

SEQ ID NO: 201 - DNA, 23 bp, synthetic construct
  source 1..23: mol_type = other DNA
gcatctgagtggctttcttctcc

SEQ ID NO: 202 - DNA, 21 bp, synthetic construct
  source 1..21: mol_type = other DNA
ctgggaacttgtccggcttta

SEQ ID NO: 203 - DNA, 28 bp, synthetic construct
  source 1..28: mol_type = other DNA
taggagtttatacatgatcctaagggca

SEQ ID NO: 204 - DNA, 26 bp, synthetic construct
  source 1..26: mol_type = other DNA
atttttggccaaacagaattgtttgc

SEQ ID NO: 205 - DNA, 21 bp, synthetic construct
  source 1..21: mol_type = other DNA
caccaaaggagctgtacctcc

SEQ ID NO: 206 - DNA, 24 bp, synthetic construct
  source 1..24: mol_type = other DNA
gtcaactccaaacaggctttttcc

SEQ ID NO: 207 - DNA, 26 bp, synthetic construct
  source 1..26: mol_type = other DNA
ttgcaaactatgtcaaggacacattt

SEQ ID NO: 208 - DNA, 28 bp, synthetic construct
  source 1..28: mol_type = other DNA
gcaaatgatctcagagttgcacaaattg

SEQ ID NO: 209 - DNA, 22 bp, synthetic construct
  source 1..22: mol_type = other DNA
gatgaacagcttggattggggc

SEQ ID NO: 210 - DNA, 25 bp, synthetic construct
  source 1..25: mol_type = other DNA
cagctgagacatagagagaggactt

SEQ ID NO: 211 - DNA, 26 bp, synthetic construct
  source 1..26: mol_type = other DNA
aacaggtcatggaagctttagatctt

SEQ ID NO: 212 - DNA, 27 bp, synthetic construct
  source 1..27: mol_type = other DNA
aaaatcctggtgataaacgtacaacct

SEQ ID NO: 213 - DNA, 23 bp, synthetic construct
  source 1..23: mol_type = other DNA
tgtcagcttcttgcgtgatagtg

SEQ ID NO: 214 - DNA, 28 bp, synthetic construct
  source 1..28: mol_type = other DNA
tttttccccaagagtctcatctattagc

SEQ ID NO: 215 - DNA, 25 bp, synthetic construct
  source 1..25: mol_type = other DNA
catgtctccttcccttggttatacc

SEQ ID NO: 216 - DNA, 27 bp, synthetic construct
  source 1..27: mol_type = other DNA
aaaaattgcaagagcaataagcatgtg

SEQ ID NO: 217 - DNA, 25 bp, synthetic construct
  source 1..25: mol_type = other DNA
atcttttgccttggcatttggtctg

SEQ ID NO: 218 - DNA, 27 bp, synthetic construct
  source 1..27: mol_type = other DNA
ctagtttgcttccttctatgaccccta

SEQ ID NO: 219 - DNA, 28 bp, synthetic construct
  source 1..28: mol_type = other DNA
gagagagtgaacagatgagaatcagaaa

SEQ ID NO: 220 - DNA, 28 bp, synthetic construct
  source 1..28: mol_type = other DNA
ttgtaatggccttgggatcaaatattct

SEQ ID NO: 221 - DNA, 25 bp, synthetic construct
  source 1..25: mol_type = other DNA
agagctttagtaagacctcagactg

SEQ ID NO: 222 - DNA, 28 bp, synthetic construct
  source 1..28: mol_type = other DNA
taaaccagactgaatgtcaaagacaaac

SEQ ID NO: 223 - DNA, 23 bp, synthetic construct
  source 1..23: mol_type = other DNA
gaataaccagtaccaggcacggc

SEQ ID NO: 224 - DNA, 25 bp, synthetic construct
  source 1..25: mol_type = other DNA
tccatccctttgagtctatgtgtcc

SEQ ID NO: 225 - DNA, 28 bp, synthetic construct
  source 1..28: mol_type = other DNA
ctcttgcttctgtcagacacttttaatt

SEQ ID NO: 226 - DNA, 25 bp, synthetic construct
  source 1..25: mol_type = other DNA
cgagtctctttttggtgtattgcca

SEQ ID NO: 227 - DNA, 25 bp, synthetic construct
  source 1..25: mol_type = other DNA
ctgggaaaccagctagaagaagaga

SEQ ID NO: 228 - DNA, 26 bp, synthetic construct
  source 1..26: mol_type = other DNA
caaacgcactgagctatttaccttag

SEQ ID NO: 229 - DNA, 26 bp, synthetic construct
  source 1..26: mol_type = other DNA
gcctccctttcagatcttacttaggt

SEQ ID NO: 230 - DNA, 27 bp, synthetic construct
  source 1..27: mol_type = other DNA
aaaatgcaacacacctaatacttcagt

SEQ ID NO: 231 - DNA, 23 bp, synthetic construct
  source 1..23: mol_type = other DNA
atgtcaggatgctccacaacggt

SEQ ID NO: 232 - DNA, 24 bp, synthetic construct
  source 1..24: mol_type = other DNA
tccacagagcatcagctatgaatc

SEQ ID NO: 233 - DNA, 25 bp, synthetic construct
  source 1..25: mol_type = other DNA
ctgatcttacaagttcacggcttgt

SEQ ID NO: 234 - DNA, 28 bp, synthetic construct
  source 1..28: mol_type = other DNA
ttctctatatgaccagcctctttacatg

SEQ ID NO: 235 - DNA, 24 bp, synthetic construct
  source 1..24: mol_type = other DNA
gctgggcatatactccaaagacag

SEQ ID NO: 236 - DNA, 27 bp, synthetic construct
  source 1..27: mol_type = other DNA
ccagtctctagtaactgtccttctctg

SEQ ID NO: 237 - DNA, 30 bp, synthetic construct
  source 1..30: mol_type = other DNA
gatattagccctttgccagatagataggtt

SEQ ID NO: 238 - DNA, 32 bp, synthetic construct
  source 1..32: mol_type = other DNA
gggaaaggattccctatttaataaatagtgtc

SEQ ID NO: 239 - DNA, 22 bp, synthetic construct
  source 1..22: mol_type = other DNA
tgtctggaaaactgtagcgtgt

SEQ ID NO: 240 - DNA, 25 bp, synthetic construct
  source 1..25: mol_type = other DNA
ccatgcacaattaggaacaacagtg

SEQ ID NO: 241 - DNA, 28 bp, synthetic construct
  source 1..28: mol_type = other DNA
gatgcgggataaggaattaatcaaggaa

SEQ ID NO: 242 - DNA, 27 bp, synthetic construct
  source 1..27: mol_type = other DNA
cactatgcctagctttgtcttgtctta

SEQ ID NO: 243 - DNA, 23 bp, synthetic construct
  source 1..23: mol_type = other DNA
gtactgcagtcacacaaagcaga

SEQ ID NO: 244 - DNA, 24 bp, synthetic construct
  source 1..24: mol_type = other DNA
ctaatgaaaggctgcctctgttct

SEQ ID NO: 245 - DNA, 24 bp, synthetic construct
  source 1..24: mol_type = other DNA
cacacgtgctagttaggctaaata

SEQ ID NO: 246 - DNA, 28 bp, synthetic construct
  source 1..28: mol_type = other DNA
cttcctttgtgacttctgttgcatttat

SEQ ID NO: 247 - DNA, 24 bp, synthetic construct
  source 1..24: mol_type = other DNA
cgctgtgaagtatttaacatgcag

SEQ ID NO: 248 - DNA, 23 bp, synthetic construct
  source 1..23: mol_type = other DNA
ggaggccttgcactgttttatga

SEQ ID NO: 249 - DNA, 26 bp, synthetic construct
  source 1..26: mol_type = other DNA
tctatggatcgttcttgcttgtttct

SEQ ID NO: 250 - DNA, 26 bp, synthetic construct
  source 1..26: mol_type = other DNA
gggctattttgttgactgagagaatg

SEQ ID NO: 251 - DNA, 28 bp, synthetic construct
  source 1..28: mol_type = other DNA
agggaaaattcttccttatgatgggaag

SEQ ID NO: 252 - DNA, 30 bp, synthetic construct
  source 1..30: mol_type = other DNA
tccagtttcaattttctgcacattgttaga

SEQ ID NO: 253 - DNA, 28 bp, synthetic construct
  source 1..28: mol_type = other DNA
tatgttgcccagaattctgagcatagac

SEQ ID NO: 254 - DNA, 26 bp, synthetic construct
  source 1..26: mol_type = other DNA
agggaggaaataattcgctttgcatt

SEQ ID NO: 255 - DNA, 24 bp, synthetic construct
  source 1..24: mol_type = other DNA
cagaatagcactggatccacaggc

SEQ ID NO: 256 - DNA, 25 bp, synthetic construct
  source 1..25: mol_type = other DNA
aagcttaattgccatgccgtttatc

SEQ ID NO: 257 - DNA, 28 bp, synthetic construct
  source 1..28: mol_type = other DNA
gtgaagacatcgtaaaaagatctacctg

SEQ ID NO: 258 - DNA, 28 bp, synthetic construct
  source 1..28: mol_type = other DNA
aatttaattgggatcaatgcccaaaagg

SEQ ID NO: 259 - DNA, 28 bp, synthetic construct
  source 1..28: mol_type = other DNA
ttattactctagaggcagggactagcct

SEQ ID NO: 260 - DNA, 25 bp, synthetic construct
  source 1..25: mol_type = other DNA
aggtatctgctgtcagtgtgactaa

SEQ ID NO: 261 - DNA, 27 bp, synthetic construct
  source 1..27: mol_type = other DNA
aagcatctttggagttctcttctccag

SEQ ID NO: 262 - DNA, 28 bp, synthetic construct
  source 1..28: mol_type = other DNA
tagacatattcctacatctgtggaatgg

SEQ ID NO: 263 - DNA, 28 bp, synthetic construct
  source 1..28: mol_type = other DNA
ctatttcaaggtaagattctgtctctta

SEQ ID NO: 264 - DNA, 30 bp, synthetic construct
  source 1..30: mol_type = other DNA
aagaacatattctaaaacagctgaatgaac

SEQ ID NO: 265 - DNA, 25 bp, synthetic construct
  source 1..25: mol_type = other DNA
aatgtcattgacgtgatcacctgca

SEQ ID NO: 266 - DNA, 21 bp, synthetic construct
  source 1..21: mol_type = other DNA
gtagtaggcgatgaagagcgt

SEQ ID NO: 267 - DNA, 25 bp, synthetic construct
  source 1..25: mol_type = other DNA
tgagttgaaaccccagtgaaacaca

SEQ ID NO: 268 - DNA, 22 bp, synthetic construct
  source 1..22: mol_type = other DNA
ccccagcaatgatctcgtaagt

SEQ ID NO: 269 - DNA, 24 bp, synthetic construct
  source 1..24: mol_type = other DNA
aacccttcctcctaacctcatatg

SEQ ID NO: 270 - DNA, 27 bp, synthetic construct
  source 1..27: mol_type = other DNA
cttacccaacagaactcaagtattggt

SEQ ID NO: 271 - DNA, 27 bp, synthetic construct
  source 1..27: mol_type = other DNA
tttacttaaagcatagcttgccttgcc

SEQ ID NO: 272 - DNA, 27 bp, synthetic construct
  source 1..27: mol_type = other DNA
cggttgcaccatttgacattctattag

SEQ ID NO: 273 - DNA, 24 bp, synthetic construct
  source 1..24: mol_type = other DNA
aacaggaaagcagatgaaactggc

SEQ ID NO: 274 - DNA, 21 bp, synthetic construct
  source 1..21: mol_type = other DNA
ctactgtgcgtgtgcgatagc

SEQ ID NO: 275 - DNA, 21 bp, synthetic construct
  source 1..21: mol_type = other DNA
tggatcacaacctcacggagg

SEQ ID NO: 276 - DNA, 26 bp, synthetic construct
  source 1..26: mol_type = other DNA
cgtcttggaagtgaaaacacatcata

SEQ ID NO: 277 - DNA, 17 bp, synthetic construct
  source 1..17: mol_type = other DNA
gatcgcagcggctacag

SEQ ID NO: 278 - DNA, 19 bp, synthetic construct
  source 1..19: mol_type = other DNA
tccatgcacagtcccacga

SEQ ID NO: 279 - DNA, 28 bp, synthetic construct
  source 1..28: mol_type = other DNA
ttcataaacaagcagatatgcaagaaga

SEQ ID NO: 280 - DNA, 25 bp, synthetic construct
  source 1..25: mol_type = other DNA
cataagccagtttcccagttttcaa

SEQ ID NO: 281 - DNA, 27 bp, synthetic construct
  source 1..27: mol_type = other DNA
ctaatgcctctgttcaagcttctttgc

SEQ ID NO: 282 - DNA, 25 bp, synthetic construct
  source 1..25: mol_type = other DNA
tcaaattcttagagctcccagctga

SEQ ID NO: 283 - DNA, 25 bp, synthetic construct
  source 1..25: mol_type = other DNA
tttccttggattccacactttgcct

SEQ ID NO: 284 - DNA, 25 bp, synthetic construct
  source 1..25: mol_type = other DNA
agtagatgggaaatcacacgcaaat

SEQ ID NO: 285 - DNA, 25 bp, synthetic construct
  source 1..25: mol_type = other DNA
gcccagtaagagctttcttttatgg

SEQ ID NO: 286 - DNA, 23 bp, synthetic construct
  source 1..23: mol_type = other DNA
gatgcacgcttatgggtagtgaa

SEQ ID NO: 287 - DNA, 21 bp, synthetic construct
  source 1..21: mol_type = other DNA
gtcttcccccaaaactgacag

SEQ ID NO: 288 - DNA, 24 bp, synthetic construct
  source 1..24: mol_type = other DNA
ggactctgaagcctctgtacacat

SEQ ID NO: 289 - DNA, 27 bp, synthetic construct
  source 1..27: mol_type = other DNA
cccaaaatattactgcagatgtcctta

SEQ ID NO: 290 - DNA, 26 bp, synthetic construct
  source 1..26: mol_type = other DNA
agcagactaatatgcctctgctattt

SEQ ID NO: 291 - DNA, 26 bp, synthetic construct
  source 1..26: mol_type = other DNA
ctcacttttcagtattctgttctgag

SEQ ID NO: 292 - DNA, 26 bp, synthetic construct
  source 1..26: mol_type = other DNA
attctgacacacaagtttatccatgc

SEQ ID NO: 293 - DNA, 27 bp, synthetic construct
  source 1..27: mol_type = other DNA
ttctccttgttcttccctgtgcatacc

SEQ ID NO: 294 - DNA, 27 bp, synthetic construct
  source 1..27: mol_type = other DNA
aagaagcttgaaagtctacagttgtcc

SEQ ID NO: 295 - DNA, 25 bp, synthetic construct
  source 1..25: mol_type = other DNA
ctttcctccccatcaatcacttggg

SEQ ID NO: 296 - DNA, 27 bp, synthetic construct
  source 1..27: mol_type = other DNA
tcaaggctatggatacctatctcttct

SEQ ID NO: 297 - DNA, 21 bp, synthetic construct
  source 1..21: mol_type = other DNA
cccagttcggcatccgtaagg

SEQ ID NO: 298 - DNA, 21 bp, synthetic construct
  source 1..21: mol_type = other DNA
ggtgcccagatatggagggaa

SEQ ID NO: 299 - DNA, 26 bp, synthetic construct
  source 1..26: mol_type = other DNA
caactagagatcaccccataactcag

SEQ ID NO: 300 - DNA, 23 bp, synthetic construct
  source 1..23: mol_type = other DNA
taaaaatgaagattcggccggac

SEQ ID NO: 301 - DNA, 26 bp, synthetic construct
  source 1..26: mol_type = other DNA
aaacagaagagcatattggccacaat

SEQ ID NO: 302 - DNA, 31 bp, synthetic construct
  source 1..31: mol_type = other DNA
gcagttaggcactaaactatattgtttcaaa

SEQ ID NO: 303 - DNA, 27 bp, synthetic construct
  source 1..27: mol_type = other DNA
cactccatcgtgaaagaataatcctgt

SEQ ID NO: 304 - DNA, 25 bp, synthetic construct
  source 1..25: mol_type = other DNA
ggttaagctgctttgaggaacaaga

SEQ ID NO: 305 - DNA, 26 bp, synthetic construct
  source 1..26: mol_type = other DNA
gaatcctaagattgtggctgagagag

SEQ ID NO: 306 - DNA, 24 bp, synthetic construct
  source 1..24: mol_type = other DNA
gttctttcctcctgacatgggaac

SEQ ID NO: 307 - DNA, 25 bp, synthetic construct
  source 1..25: mol_type = other DNA
ccaaaagtaagaactgcttcaggga

SEQ ID NO: 308 - DNA, 27 bp, synthetic construct
  source 1..27: mol_type = other DNA
atgagccacattactttgttttctagg

SEQ ID NO: 309 - DNA, 25 bp, synthetic construct
  source 1..25: mol_type = other DNA
tgtggtgatgactgagagatgatgc

SEQ ID NO: 310 - DNA, 22 bp, synthetic construct
  source 1..22: mol_type = other DNA
ccatagaccagtggatgccaac

SEQ ID NO: 311 - DNA, 23 bp, synthetic construct
  source 1..23: mol_type = other DNA
tgtgagtcctctcggtcatagca

SEQ ID NO: 312 - DNA, 26 bp, synthetic construct
  source 1..26: mol_type = other DNA
catggcattatcagggtctgaagaaa

SEQ ID NO: 313 - DNA, 24 bp, synthetic construct
  source 1..24: mol_type = other DNA
aaagcttatgtggtaggagcctaa

SEQ ID NO: 314 - DNA, 27 bp, synthetic construct
  source 1..27: mol_type = other DNA
caacaagagaggacaaattctttcaca

SEQ ID NO: 315 - DNA, 26 bp, synthetic construct
  source 1..26: mol_type = other DNA
gtacctagcttagggttagacatctg

SEQ ID NO: 316 - DNA, 27 bp, synthetic construct
  source 1..27: mol_type = other DNA
tgtgcagaaataacagagtgagaaagt

SEQ ID NO: 317 - DNA, 25 bp, synthetic construct
  source 1..25: mol_type = other DNA
gaagtccaaagtcaaagtgtcagca

SEQ ID NO: 318 - DNA, 32 bp, synthetic construct
  source 1..32: mol_type = other DNA
tgttttggatgatatgtttccttttgttcatt

SEQ ID NO: 319 - DNA, 24 bp, synthetic construct
  source 1..24: mol_type = other DNA
agaggagcttcacaaacatccgct

SEQ ID NO: 320 - DNA, 23 bp, synthetic construct
  source 1..23: mol_type = other DNA
ctgctggtgaatcagcaaaacct

SEQ ID NO: 321 - DNA, 21 bp, synthetic construct
  source 1..21: mol_type = other DNA
ttatcggctggaacgagttca

SEQ ID NO: 322 - DNA, 25 bp, synthetic construct
  source 1..25: mol_type = other DNA
ggtgataacagcttctcctgtaagg

SEQ ID NO: 323 - DNA, 19 bp, synthetic construct
  source 1..19: mol_type = other DNA
cgtggacgccgtgattcag

SEQ ID NO: 324 - DNA, 24 bp, synthetic construct
  source 1..24: mol_type = other DNA
gtgatagtgggttttcagtgaacg

SEQ ID NO: 325 - DNA, 24 bp, synthetic construct
  source 1..24: mol_type = other DNA
gagccaatcttcagtcagtaccgc

SEQ ID NO: 326 - DNA, 22 bp, synthetic construct
  source 1..22: mol_type = other DNA
cctgtggtcacagttcttggtc

(19) United States
(12) Patent Application Publication
(10) Pub. No.: US 2023/0212671 A1
(43) Pub. Date: Jul. 6, 2023

(54) PRIMER COMPOSITION, KIT AND METHOD FOR DETECTING MICROHAPLOTYPE LOCI BASED ON NEXT GENERATION SEQUENCING TECHNOLOGY, AND APPLICATIONS THEREOF

(71) Applicants: Fudan University, Shanghai (CN); Academy of Forensic Science, Shanghai (CN)

(72) Inventors: SuHua Zhang, Shanghai (CN); ChengTao Li, Shanghai (CN); AnQi Chen, Shanghai (CN); RuiYang Tao, Shanghai (CN)

(21) Appl. No.: 18/067,693

(22) Filed: Dec. 16, 2022

Related U.S. Application Data
(63) Continuation of application No. PCT/CN2022/115540, filed on Aug. 29, 2022.

(30) Foreign Application Priority Data
Aug. 30, 2021 (CN) 2021110077419

(51) Int. Cl. C12Q 1/6876; C12Q 1/6869; C12Q 1/6858; C12Q 1/686; C12Q 1/6855

(52) U.S. Cl. CPC C12Q 1/6876; C12Q 1/6869; C12Q 1/6858; C12Q 1/686; C12Q 1/6855; C12Q 2600/16

(57) ABSTRACT

A primer composition, a kit and a method for detecting microhaplotype loci based on next generation sequencing technology and applications thereof are provided, relating to the technical field of forensic medicine, which are used to amplify 163 microhaplotype loci on human genome. The primer composition includes one or more pairs of primers with sequences as shown in SEQ ID NO: 1˜326. The primer composition involves 163 microhaplotype loci covering 22 autosomes, which can provide more new genetic information in Asian population than the system constructed in the past. In addition, compared with the next generation sequencing kit of STR loci, the kit has better mixture detection capability. Moreover, the microhaplotype genetic markers have high ancestry information content and can distinguish populations in Africa, Europe, South Asia, and East Asia. Therefore, the microhaplotype genetic markers can also be used for ancestry inference in addition to individual identification and parentage testing.

### TECHNICAL FIELD

The invention relates to forensic technology, more particular to a primer composition, a kit and a method for detecting microhaplotype (MH) loci based on next generation sequencing technology, and applications thereof. The primer composition is used for amplifying 163 microhaplotype loci covering 22 pairs of autosomes (also referred to as hetero chromosomes).

### STATEMENT REGARDING SEQUENCE LISTING

The sequence listing associated with this application is provided in text format in lieu of a paper copy and is hereby incorporated by reference into the specification. The name of the XML file containing the sequence listing is 22033THXT-USP1-US-2022-0034-SL.xml. The XML file is 290,816 bytes; was created on Sep. 28, 2022; contains no new matter; and is being submitted electronically via EFS-Web.

### BACKGROUND

Forensic genetics mainly relies on the detection and analysis of deoxyribonucleic acid (DNA) genetic markers to solve problems related to individual identification and parentage testing in judicial practice. Among many kinds of genetic markers, short tandem repeat (STR) is the most commonly used genetic marker because of its good polymorphism and simple typing method. Biallelic single nucleotide polymorphism (SNP) and insertion-deletion (InDel) markers have the advantages of low mutation rate and short amplification fragment, which can make up for the shortcomings of STR of high mutation rate, large amplification fragment and stutter peaks in typing, and have more advantages in the analysis of degradation samples and biogeographic ancestry inference. However, due to the low polymorphism of a single locus, it is often necessary to increase the number of detection loci to achieve the detection efficiency similar to that of the STR system. Therefore, some scholars proposed the concept of compound genetic markers, including linked genetic markers SNP-STR, InDel-STR, multi-InDel, etc.

In 2014, Professor Kenneth K. Kidd (“Current sequencing technology makes microhaplotypes a powerful new type of genetic marker for forensics”, Forensic Science International: Genetics, 2014, pp 215-224) of Yale University proposed the concept of microhaplotype (MH), which is a locus with two or more SNP sites within a 200-300 base pair (bp) DNA segment. Microhaplotypes composed of SNPs not only have high polymorphism comparable to STR loci and do not produce stutter peaks, but also retain the characteristics of low mutation rate and short fragments of SNPs, which have advantages in forensic community. Some systems including microhaplotype markers (also referred to as microhaplotype loci), such as a compound system with 74 microhaplotype markers constructed by Oldoni et al. (“A sequence-based 74plex microhaplotype assay for analysis of forensic DNA mixtures”, Forensic Science International: Genetics, 2020, page 102367) and a compound system with 118 microhaplotype markers constructed by Maria de la Puente et al. (“Building a custom large-scale panel of novel microhaplotypes for forensic identification using MiSeq and Ion S5 massively parallel sequencing systems”, Forensic Science International: Genetics, 2020, page 102213), have good capabilities of individual identification, parentage testing, and mixture analysis.

For the analysis and detection of mixture samples, the traditional STR typing test often shows multiple allele peaks. It is difficult to distinguish stutter peaks from the allele peaks with a small contribution ratio or noise allele, and the interpretation of the evidence value is quite difficult. MH has no stutter peak interference, advantages of both STR and SNP markers, which is an ideal genetic marker for analysis and detection of the mixture samples.

Due to long-term migration and evolution, the frequency distribution of some SNPs varies greatly among different populations. Screening MH composed of ancestry-informative SNP (AI-SNP) can provide an important basis for research on population structure and ancestry inference in forensic community. Kenneth K. Kidd initially established a system containing 31 MH markers, which can better distinguish the five major geographical regions of Africa, Europe, Southeast Asia, East Asia, America and Pacific islands, showing the superiority of MH as an ancestral information marker.

Next generation sequencing (NGS), also known as massively parallel sequencing, has the advantages of high throughput and high accuracy, which provides a platform for the detection and application of new genetic markers. MH is composed of multiple SNPs, which is essentially sequence polymorphism. The next generation sequencing can obtain all MH typing at one time, realize the parallel analysis and detection of a large number of genetic markers.

### SUMMARY

In order to overcome the defects in the related art, the invention screens MH loci with forensic application value in ancestry inference, mixture analysis, individual identification and parentage testing in Asian population, and develops and establishes a primer composition and a kit that can simultaneously detect 163 MH loci at a time based on next generation sequencing technology.

To achieve the above purpose, the invention adopts the following technical solutions as follows.

In a first aspect of the invention, a primer composition for detecting MH loci based on the next generation sequencing technology is provided. The primer composition includes one or more pairs of amplification primers of 163 MH loci.

The 163 MH loci consist of mh01CP007, mh01CP008, mh01CP012, mh01CP016, mh01KK001, mh01KK070, mh01KK072, mh01KK106, mh01KK117, mh01KK172, mh01KK205, mh01KK210, mh01KK211, mh02CP004, mh02KK003, mh02KK004, mh02KK073, mh02KK102, mh02KK105, mh02KK131, mh02KK134, mh02KK136, mh02KK138, mh02KK139, mh02KK201, mh02KK202, mh02KK213, mh02KK215, mh03KK006, mh03KK007, mh03KK008, mh03KK009, mh03KK216, mh04CP002, mh04CP003, mh04CP007, mh04KK010, mh04KK011, mh04KK013, mh04KK015, mh04KK016, mh04KK017, mh04KK019, mh04KK028, mh04KK029, mh04KK030, mh04KK074, mh05CP004, mh05CP006, mh05CP010, mh05KK020, mh05KK022, mh05KK062, mh05KK078, mh05KK079, mh05KK122, mh05KK123, mh05KK124, mh05KK170, mh06CP003, mh06CP007, mh06KK026, mh06KK030, mh06KK031, mh06KK080, mh06KK101, mh07KK030, mh07KK031, mh07KK081, mh07KK082, mh08KK032, mh09KK020, mh09KK033, mh09KK034, mh09KK152, mh09KK153, mh09KK157, mh09KK161, mh10CP003, mh10KK083, mh10KK084, mh10KK085, mh10KK086, mh10KK087, mh10KK088, mh10KK101, mh10KK163, mh10KK170, mh11CP003, mh11CP004, mh11CP005, mh11KK036, mh11KK037, mh11KK038, mh11KK039, mh11KK040, mh11KK041, mh11KK089, mh11KK090, mh11KK091, mh11KK180, mh11KK187, mh11KK191, mh12KK042, mh12KK043, mh12KK045, mh12KK046, mh12KK092, mh12KK093, mh12KK202, mh13CP008, mh13KK047, mh13KK213, mh13KK217, mh13KK218, mh13KK225, mh13KK226, mh14CP003, mh14CP004, mh14KK048, mh14KK101, mh15CP001, mh15CP003, mh15CP004, mh15KK066, mh15KK067, mh15KK069, mh15KK095, mh16KK053, mh16KK062, mh16KK096, mh16KK255, mh16KK302, mh17CP001, mh17CP006, mh17KK014, mh17KK052, mh17KK053, mh17KK054, mh17KK055, mh17KK077, mh17KK105, mh17KK110, mh17KK272, mh18CP003, mh18CP005, mh18KK285, mh18KK293, mh19CP007, mh19KK056, mh19KK057, mh19KK299, mh19KK301, mh20KK058, mh20KK059, mh20KK307, mh21KK313, mh21KK315, mh21KK316, mh21KK324, mh22KK060, mh22KK064, and mh22KK303.

In an embodiment, the primer composition includes one or more pairs of the amplification primers with nucleotide sequences respectively shown in SEQ ID NO: 1 through SEQ ID NO: 326.

In an embodiment, the primer composition includes the amplification primers with the nucleotide sequences respectively shown in SEQ ID NO: 1 to SEQ ID NO: 326.

In a second aspect of the invention, a kit for detecting MH loci based on the next generation sequencing technology including the primer composition is provided, and the kit further includes a polymerase chain reaction (PCR) mixed solution and a PCR reaction solution.

In a third aspect of the invention, a method for detecting MR loci based on the next generation sequencing technology using the kit above is provided, including the following steps:

step 1, taking a sample to be tested, extracting a DNA sample, and quantifying extracted DNA sample;

step 2, preparing a multiplex PCR system, and conducting a first round of multiplex PCR; after a reaction of the first round of multiplex PCR is completed, obtaining a product, then adding a purification reaction solution to purify the product, and conducting magnetic bead sorting on the purified product;

step 3, repairing the purified product to make ends equal and adding an adenine base (A) into the ends then ligating sequencing adapters on the ends to obtain a complemented product, and then purifying the complemented product again using purification magnetic beads to obtain a purified elution product;

step 4, conducting a PCR reaction on the purified elution product using a reaction system to construct a library, wherein the reaction system includes the elution purified product, a PCR mixed solution, a QU reagent, a mixed post-P5 primer, and a mixed pre-p7 primer;

step 5, conducting purification and quantification on the library, specifically including: purifying the product by using purification magnetic beads, and conducting quantification and quality control on the library by using Qubit™;

step 6, conducting sequencing and data analysis, specifically including: using the constructed library on a MiSeq FGx™ platform for sequencing to obtain sequencing data; trimming the sequencing adapters of the obtained sequencing data by using a Trimmatic software to obtain sequences, then comparing the sequences with human reference genome hg19 by using a burrows-wheeler aligner (BWA) software, and obtaining MH typing by using a Python tool.

In an embodiment, a concentration of the DNA sample is 5 nanograms per microliter (ng/μL).

In an embodiment, the multiplex PCR system includes 20 μL total reaction volume, specifically including 8 μL of the PCR mixed solution, 2 μL of the PCR reaction solution, 8 μL of primer mixed solution, and 2 μL of the DNA sample.

In an embodiment, a concentration of the primer mixed solution is 0.5 micromoles per liter (μM).

In an embodiment, reaction conditions of the multiplex PCR in the step 2 includes: pre-denaturation at 95° C. for 15 minutes; denaturation at 95° C. for 30 seconds, annealing at 60° C. for 90 seconds, extension at 72° C. for 30 seconds, 24 cycles, heat preservation at 72° C. for 10 minutes.

In an embodiment, a reaction system of the repairing the purified product to make ends equal and adding A into the ends in the step 3 includes 50 μL total reaction volume, specifically including 42 μL of the purified product in the step 2, 6.8 μL of end repair dA-tailing buffer, and 1.2 μL of end repair dA-tailing enzyme.

In an embodiment, reaction conditions of the repairing the purified product to make ends equal and adding A into the ends in the step 3 includes: reaction at 30° C. for 30 minutes, then reaction at 65° C. for 30 minutes, and finally heat preservation at 4° C.

In an embodiment, a reaction system of the ligating sequencing adapters in the step 3 includes 80 μL total reaction volume, specifically including 50 μL of the purified elution product in the step 3, 2.5 μL of adapter mixed solution, 16 μL of ligation buffer, 10 μL of ligase, and 1.5 μL of nuclease-free water.

In an embodiment, reaction conditions of the ligating sequencing adapters in the step 3 includes: reaction at 25° C. for 15 minutes, and heat preservation at 4° C.

In an embodiment, a reaction system of the PCR reaction of the step 4 includes 50 μL total reaction volume, specifically including 14 μL of the elution purified product of the step 3, 25 μL of the PCR mixed solution, 3 μL of the QU reagent, 5μL of the mixed capture post-P5 primer, and 5 μL of the mixed capture pre-p7 primer.

In an embodiment, reaction conditions of the PCR reaction in the step 4 includes: reaction at 37° C. for 15 minutes; pre-denaturation at 98° C. for 45 seconds; denaturation at 98° C. for 15 seconds, annealing at 60° C. for 30 seconds, extension at 72° C. for 30 seconds, 10 cycles, then reaction at 72° C. for 5 minutes, and heat preservation at 4° C.

In a fourth aspect of the invention, an application/use of the primer composition or the kit in individual identification, parentage testing, mixture analysis and ancestry inference.

In an embodiment, the individual identification and parentage testing are mainly based on typing results of 48 MH loci with good polymorphism. The 48 MH loci consist of: mh01CP008, mh01CP012, mh01CP016, mh01KK117, mh01KK205, mh01KK211, mh02KK134, mh02KK136, mh04CP002, mh04CP003, mh04CP007, mh04KK030, mh05CP004, mh05CP006, mh05KK020, mh05KK170, mh06CP003, mh06CP007, mh09KK153, mh10CP003, mh10KK163, mh11CP003, mh11CP005, mh11KK180, mh12KK046, mh12KK202, mh13CP008, mh13KK213, mh13KK217, mh13KK218, mh13KK225, mh14CP003, mh14CP004, mh15CP001, mh15KK066, mh16KK255, mh16KK302, mh17CP001, mh17CP006, mh17KK272, mh18CP003, mh18CP005, mh19CP007, mh19KK299, mh20KK058, mh20KK307, mh21KK315, and mh21KK324.

In an embodiment, the genomic DNA sample extracted from the biological sample or the mixed biological sample is subjected to library construction, purification and quantification by using the primer composition, and the constructed library is placed on a MiSeq FGx™ platform for sequencing analysis, and finally the obtained sequencing data is analyzed to obtain the MH typing.

The invention adopts the above technical solutions and has the following technical effects compared with the related art.

The primer composition for detecting MH loci based on the next generation sequencing technology provided by the invention involves 163 MH loci covering 22 pairs of autosomes, which can provide more new genetic information than the system constructed in the past. In addition, compared with the next generation sequencing kit of STR loci, the kit of the invention has better mixture detection capability. Moreover, the MH loci involved in the invention have high ancestry information content and can distinguish populations in Africa, Europe, South Asia and East Asia.

### BRIEF DESCRIPTION OF DRAWINGS

FIG. 1 illustrates statistics of sequencing results of DNA with different concentration gradients detected by a method according to embodiment 1 of the invention.

FIG. 2 illustrates results of sequencing uniformity of DNA with different concentration gradients detected by the method according to the embodiment 1 of the invention.

FIG. 3 illustrates principal component analysis results in 27 populations worldwide detected by the method according to the embodiment 1 of the invention.

### DETAILED DESCRIPTION OF EMBODIMENTS

The invention relates to a primer composition for detecting microhaplotype (MH) loci (also referred to as MH markers or MH) based on a next generation sequencing technology, and the primer composition includes one or more pairs of amplification primers of 163 MH loci.

Specifically, the 163 MHs are all from MH loci included in ALFRED website and MHs published in literature, distributed in intron regions, with good polymorphism in Asian population, and a distribution length being smaller or equal than 300 bp. Names, chromosome information and locus information of the 163 MH loci are shown in Table 1:

TABLE 1
Name, chromosome information and
SNP information of 163 MH loci

| No | Microhaplotype | Chr | SNP |
|---|---|---|---|
| 1 | mh01CP007 | 1 | rs74887893/rs80137938/rs861907 |
| 2 | mh01CP008 | 1 | rs10803282/rs10803283/rs10927447 |
| 3 | mh01CP012 | 1 | rs12026749/rs1283256/rs8179472 |
| 4 | mh01CP016 | 1 | rs11206620/rs4927251/rs6684891 |
| 5 | mh01KK001 | 1 | rs4648344/rs58111155/rs6663840/ |
| | | | rs6688969 |
| 6 | mh01KK070 | 1 | rs1801131/rs4846051 |
| 7 | mh01KK072 | 1 | rs1251078/rs1251079 |
| 8 | mh01KK106 | 1 | rs12123330/rs16840876/rs4468133/ |
| | | | rs56212601 |
| 9 | mh01KK117 | 1 | rs1610400/rs1610401/rs17413714/ |
| | | | rs2772234 |
| 10 | mh01KK172 | 1 | rs1887284/rs3128342/rs3766176 |
| 11 | mh01KK205 | 1 | rs11810587/rs1336130/rs1533622/ |
| | | | rs1533623 |
| 12 | mh01KK210 | 1 | rs2165332/rs7536195 |
| 13 | mh01KK211 | 1 | rs16835l27/rs2341465/rs2490423 |
| 14 | mh02CP004 | 2 | rs4668522/rs4669133/rs55990245 |
| 15 | mh02KK003 | 2 | rs11123719/rs11691107/rs260694 |
| 16 | mh02KK004 | 2 | rs13424991/rs3731611/rs3731612 |
| 17 | mh02KK073 | 2 | rs1374748/rs7583554 |
| 18 | mh02KK102 | 2 | rs2169812/rs2378217/rs6542783 |
| 19 | mh02KK105 | 2 | rs2280355/rs2280356 |
| 20 | mh02KK131 | 2 | rs1466020/rs17488897 |
| 21 | mh02KK134 | 2 | rs12469721/rs3101043/rs3111398/ |
| | | | rs72623112 |
| 22 | mh02KK136 | 2 | rs12617010/rs6714835/rs6756898 |
| 23 | mh02KK138 | 2 | rs2595202/rs2595203/rs4953292/ |
| | | | rs59298278/rs6715568/rs6759301 |
| 24 | mh02KK139 | 2 | rs12623957/rs3827760 |
| 25 | mh02KK201 | 2 | rs1371048/rs786247 |
| 26 | mh02KK202 | 2 | rs12464185/rs13422174 |
| 27 | mh02KK213 | 2 | rs1519654/rs7568519/rs7577785 |
| 28 | mh02KK215 | 2 | rs16832624/rs2011946 |
| 29 | mh03KK006 | 3 | rs1919550/rs9873644 |
| 30 | mh03KK007 | 3 | rs4513489/rs6441961 |
| 31 | mh03KK008 | 3 | rs17030627/rs6808142 |
| 32 | mh03KK009 | 3 | rs3732783/rs6280 |
| 33 | mh03KK216 | 3 | rs1046953/rs2072053 |
| 34 | mh04CP002 | 4 | rs34017818/rs35619595/rs6814654 |
| 35 | mh04CP003 | 4 | rs10006433/rs29801189/rs58595616 |
| 36 | mh04CP007 | 4 | rs4697751/rs4698039/rs4698040 |
| 37 | mh04KK010 | 4 | rs3135123/rs495367 |
| 38 | mh04KK011 | 4 | rs6531591/rs6855439 |
| 39 | mh04KK013 | 4 | rs11725922/rs13131164/rs17088476/ |
| | | | rs3775866/rs3775867 |
| 40 | mh04KK015 | 4 | rs12648443/rs2584457 |
| 41 | mh04KK016 | 4 | rs2032350/rs2851017 |
| 42 | mh04KK017 | 4 | rs1442492/rs2584461/rs4699748 |
| 43 | mh04KK019 | 4 | rs17731793/rs2122136 |
| 44 | mh04KK028 | 4 | rs283413/rs3762896 |
| 45 | mh04KK029 | 4 | rs59534319/rs971074 |
| 46 | mh04KK030 | 4 | rs16844737/rs1884411/rs1884412/ |
| | | | rs4916615 |
| 47 | mh04KK074 | 4 | rs11932595/rs17085763 |
| 48 | mh05CP004 | 5 | rs150628/rs16883189/rs61243436 |
| 49 | mh05CP006 | 5 | rs12653673/rs6555064/rs6555065 |
| 50 | mh05CP010 | 5 | rs62349578/rs62349579/rs62349580/ |
| | | | rs62349581 |
| 51 | mh05KK020 | 5 | rs2278324/rs2278325/rs525735/ |
| | | | rs617938 |
| 52 | mh05KK022 | 5 | rs41461/rs41462 |
| 53 | mh05KK062 | 5 | rs870347/rs870348 |
| 54 | mh05KK078 | 5 | rs2234233/rs2234234 |
| 55 | mh05KK079 | 5 | rs2234232/rs41469 |
| 56 | mh05KK122 | 5 | rs1010872/rs28777 |
| 57 | mh05KK123 | 5 | rs1423676/rs28117 |
| 58 | mh05KK124 | 5 | rs35414/rs3756464 |
| 59 | mh05KK170 | 5 | rs370672/rs438055/rs6555108/ |
| | | | rs74865590 |
| 60 | mh06CP003 | 6 | rs12202010/rs4960100/rs4960101 |
| 61 | mh06CP007 | 6 | rs4142082/rs558006/rs6906397 |
| 62 | mh06KK026 | 6 | rs179939/rs4431439/rs4565296 |
| 63 | mh06KK030 | 6 | rs10949381/rs607341/rs675934 |
| 64 | mh06KK031 | 6 | rs10455681/rs10455682 |
| 65 | mh06KK080 | 6 | rs2056941/rs2056942 |
| 66 | mh06KK101 | 6 | rs2180052/rs9356632 |
| 67 | mh07KK030 | 7 | rs10226425/rs2330425/rs967066 |
| 68 | mh07KK031 | 7 | rs10246622/rs17168174 |
| 69 | mh07KK081 | 7 | rs28365094/rs41303343 |
| 70 | mh07KK082 | 7 | rs150209521/rs713598 |
| 71 | mh08KK032 | 8 | rs1390950/rs2898295 |
| 72 | mh09KK020 | 9 | rs10810635/rs10962598/rs10962599/ |
| | | | rs73649032 |
| 73 | mh09KK033 | 9 | rs10815466/rs17431629/rs9408671 |
| 74 | mh09KK034 | 9 | rs1408800/rs1408801 |
| 75 | mh09KK152 | 9 | rs10780576/rs10867949/rs4282648/ |
| | | | rs7046769 |
| 76 | mh09KK153 | 9 | rs10125791/rs2987741/rs7047561 |
| 77 | mh09KK157 | 9 | rs2073578/rs56256724/rs606141/ |
| | | | rs633153/rs8193001 |
| 78 | mh09KK161 | 9 | rs16932430/rs4741823 |
| 79 | mh10CP003 | 10 | rs10764460/rs220365/rs727269 |
| 80 | mh10KK083 | 10 | rs11568732/rs12248560 |
| 81 | mh10KK084 | 10 | rs1058930/rs11572103 |
| 82 | mh10KK085 | 10 | rs11572076/rs2275622 |
| 83 | mh10KK086 | 10 | rs17110453/rs7909236 |
| 84 | mh10KK087 | 10 | rs10884095/rs1452267 |
| 85 | mh10KK088 | 10 | rs2515641/rs55897648 |
| 86 | mh10KK101 | 10 | rs915907/rs915908 |
| 87 | mh10KK163 | 10 | rs3814588/rs3814589/rs3814590/ |
| | | | rs6602026/rs9423466 |
| 88 | mh10KK170 | 10 | rs12359688/rs2250840/rs2250841 |
| 89 | mh11CP003 | 11 | rs12289831/rs2045045/rs2045046 |
| 90 | mh11CP004 | 11 | rs35728001/rs76882177/rs77516091 |
| 91 | mh11CP005 | 11 | rs7118419/rs72865222/rs7926642 |
| 92 | mh11KK036 | 11 | rs10500616/rs249993693 |
| 93 | mh11KK037 | 11 | rs10898849/rs341065/rs395447 |
| 94 | mh11KK038 | 11 | rs2303377/rs2303378 |
| 95 | mh11KK039 | 11 | rs10891537/rs2288159 |
| 96 | mh11KK040 | 11 | rs11214596/rs4938013 |
| 97 | mh11KK041 | 11 | rs6275/rs6277 |
| 98 | mh11KK089 | 11 | rs1124492/rs1124493 |
| 99 | mh11KK090 | 11 | rs1079597/rs1079598 |
| 100 | mh11KK091 | 11 | rs1799732/rsl799978 |
| 101 | mh11KK180 | 11 | rs12802112/rs28631755/rs4752777/ |
| | | | rs7112918 |
| 102 | mh11KK187 | 11 | rs17137917/rs17137926/rs493442/ |
| | | | rs551850 |
| 103 | mh11KK191 | 11 | rs12289401/rs12420819/rs12421109/ |
| | | | rs770566 |
| 104 | mh12KK042 | 12 | rs593226/rs7969300 |
| 105 | mh12KK043 | 12 | rs11062734/rs11613749/rs17780102 |
| 106 | mh12KK045 | 12 | rs2133298/rs3817446 |
| 107 | mh12KK046 | 12 | rs11068953/rs1503767 |
| 108 | mh12KK092 | 12 | rs2707209/rs2857234 |
| 109 | mh12KK093 | 12 | rs11111391/rs7970874 |
| 110 | mh12KK202 | 12 | rs10506052/rs10506053/rs4931233/ |
| | | | rs4931234 |
| 111 | mh13CP008 | 13 | rs9507311/rs9553248/rs9553249 |
| 112 | mh13KK047 | 13 | rs2066700/rs806301 |
| 113 | mh13KK213 | 13 | rs679482/rs8181845/rs9510616 |
| 114 | mh13KK217 | 13 | rs2765614/rs7320507/rs9562648/ |
| | | | rs9562649 |
| 115 | mh13KK218 | 13 | rs1927847/rs7492234/rs9536429/ |
| | | | rs9536430 |
| 116 | mh13KK225 | 13 | rs4884651/rs7329287/rs9529023 |
| 117 | mh13KK226 | 13 | rs2892698/rs721367 |
| 118 | mh14CP003 | 14 | rs12436504/rs66481544/rs7155003 |
| 119 | mh14CP004 | 14 | rs11157032/rs11157033/rs11157034 |
| 120 | mh14KK048 | 14 | rs12717560/rs12878166 |
| 121 | mh14KK101 | 14 | rs10134526/rs28529526 |
| 122 | mh15CP001 | 15 | rs12899727/rs34090207/rs369577479 |
| 123 | mh15CP003 | 15 | rs12440416/rs578662/rs58022506 |
| 124 | mh15CP004 | 15 | rs28628574/rs34306395/rs506120 |
| 125 | mh15KK066 | 15 | rs1063902/rs4219 |
| 126 | mh15KK067 | 15 | rs701463/rs701464 |
| 127 | mh15KK069 | 15 | rs1800410/rs1900758 |
| 128 | mh15KK095 | 15 | rs2433354/rs2459391 |
| 129 | mh16KK053 | 16 | rs11150606/rs201075024 |
| 130 | mh16KK062 | 16 | rs28485311/rs28503604/rs8055777 |
| 131 | mh16KK096 | 16 | rs1805007/rs885479 |
| 132 | mh16KK255 | 16 | rs16956011/rs3934955/rs3934956/ |
| | | | rs4073828 |
| 133 | mh16KK302 | 16 | rs1395579/rs1395580/rs1395582/ |
| | | | rs9939248 |
| 134 | mh17CP001 | 17 | rs36040276/rs4792125/rs62063465 |
| 135 | mh17CP006 | 17 | rs2215237/rs62069897/rs9897281 |
| 136 | mh17KK014 | 17 | rs11657785/rs333113/rs8074965 |
| 137 | mh17KK052 | 17 | rs1059504/rs8327 |
| 138 | mh17KK053 | 17 | rs3760370/rs3760371 |
| 139 | mh17KK054 | 17 | rs2233362/rs634370 |
| 140 | mh17KK055 | 17 | rs11868709/rs9907137 |
| 141 | mh17KK077 | 17 | rs4074461/rs4074462 |
| 142 | mh17KK105 | 17 | rs1052553/rs11568305/rs17652121 |
| 143 | mh17KK110 | 17 | rs8075367/rs9908046 |
| 144 | mh17KK272 | 17 | rs16955257/rs2934897/rs7207239/ |
| | | | rs7212184 |
| 145 | mh18CP003 | 18 | rs12970683/rs58533252/rs78549053 |
| 146 | mh18CP005 | 18 | rs595107/rs62085085/rs690302/ |
| | | | rs77849214 |
| 147 | mh18KK285 | 18 | rs16940823/rs17187688/rs17187695/ |
| | | | rs1945150 |
| 148 | mh18KK293 | 18 | rs621320/rs621340/rs621766/rs678179 |
| 149 | mh19CP007 | 19 | rs10417429/rs10417450/rs34190726 |
| 150 | mh19KK056 | 19 | rs1055919/rs2271057 |
| 151 | mh19KK057 | 19 | rs12462026/rs17717333/rs7250849 |
| 152 | mh19KK299 | 19 | rs12985452/rs2361019/rs2860462/ |
| | | | rs4932769/rs4932999 |
| 153 | mh19KK301 | 19 | rs10408037/rs10408594/rs11084040/ |
| | | | rs8104441 |
| 154 | mh20KK058 | 20 | rs601288/rs6095836/rs6122890 |
| 155 | mh20KK059 | 20 | rs10854214/rs10854215 |
| 156 | mh20KK307 | 20 | rs16997830/rs17674942/rs6044080/ |
| | | | rs6044081 |
| 157 | mh21KK313 | 21 | rs6586324/rs6586325/rs6586326 |
| 158 | mh21KK315 | 21 | rs6517971/rs8126597/rs8131148 |
| 159 | mh21KK316 | 21 | rs17002090/rs2830208/rs961301/ |
| | | | rs961302 |
| 160 | mh21KK324 | 21 | rs2838868/rs6518223/rs7279250/ |
| | | | rs8133697 |
| 161 | mh22KK060 | 22 | rs4680/rs4818 |
| 162 | mh22KK064 | 22 | rs136177/rs60910145/rs71785313/ |
| | | | rs73885319 |
| 163 | mh22KK303 | 22 | rs4633/rs6267/rs740602/rs76452330 |

Multiplex PCR primers are designed according to physical locations. Design principles include: (1) an optimal melting temperature; (2) avoidance of primer dimers and hairpin structures; (3) guanine and cytosine bases (GC) content between 20% and 80%; (4) off-target analysis to reduce primer off-target hybridization; and (5) overlap analysis to reduce the number of primers. In an embodiment of the invention, the primer composition includes one or more pairs of primers with nucleotide sequences shown in SEQ ID NO: 1 through SEQ ID NO: 326. Specific primer sequence information is shown in Table 2:

TABLE 2
Amplification primer sequence numbers and primer sequences for 163
MH loci

| Microhaplotype | Sequence number | Primer sequence |
|---|---|---|
| mh01CP007 | SEQ ID NO: 1 | TTCTCCCCAAATCACAGCACCC |
| | SEQ ID NO: 2 | CGTAAGGATGGGCAAAACGTTCA |
| mh01CP008 | SEQ ID NO: 3 | AAGCAGTTTGATGTGAGCTCTAAAACG |
| | | A |
| | SEQ ID NO: 4 | GCCAGTAGAAATTCTAAAACAAAACCC |
| | | A |
| mh01CP012 | SEQ ID NO: 5 | ATCATTTTCTCAGTGCGCAACAC |
| | SEQ ID NO: 6 | CTTTGATGTCAGATTTTCTTAGGACCGA |
| mh01CP016 | SEQ ID NO: 7 | CACTCACTTTGTGACCATTCCGGT |
| | SEQ ID NO: 8 | CTGAAGGACTACTACCTCTTCTACCT |
| mh01KK001 | SEQ ID NO: 9 | GATGAGCACCTCGAGAAGACCT |
| | SEQ ID NO: 10 | GATGGCTGGTACCGATCATCTC |
| mh01KK070 | SEQ ID NO: 11 | TAGCAACGCCAATCTCAGAGAGGT |
| | SEQ ID NO: 12 | TGCTGTAAGCACTCTACACATATCAATT |
| mh01KK072 | SEQ ID NO: 13 | ATAAGCTATGCTGAGGGAAGTCTGG |
| | SEQ ID NO: 14 | ATGAAGCTGGCTCAGTCAACTC |
| mh01KK106 | SEQ ID NO: 15 | CATAGTTTCCAGAGTGGTTTGCAGGC |
| | SEQ ID NO: 16 | ATGAGATGGGTGGTGGACAGTTA |
| mh01KK117 | SEQ ID NO: 17 | TCCTAGGCGTAAATGGATGAGAG |
| | SEQ ID NO: 18 | ATGATAGAATGTAGAACCCAGCCATC |
| mh01KK172 | SEQ ID NO: 19 | CTTAATGATAATGCTGGCAGAGTCTG |
| | SEQ ID NO: 20 | TTGATATATTTCCAAACACCTGTGTGCT |
| mh01KK205 | SEQ ID NO: 21 | ATCTTTAAGAGTCCGCTITGTGTTT |
| | SEQ ID NO: 22 | AATGTCTCCCTGAGGAATTCTACCT |
| mh01KK210 | SEQ ID NO: 23 | GCAAGATACCAAGTTCTTGAATAAGGA |
| | | G |
| | SEQ ID NO: 24 | CACCTCCTCCATAATCCACAAGTG |
| mh01KK211 | SEQ ID NO: 25 | CACAAAATGAGAGGAAGGTTACTGAG |
| | SEQ ID NO: 26 | CAAAGGAGGTCACATCACCATCTC |
| mh02CP004 | SEQ ID NO: 27 | GAATCTACTTCACTTGAATGCATGTTAA |
| | SEQ ID NO: 28 | GGAGAAACTAAGCCATATATCCATGGT |
| mh02KK003 | SEQ ID NO: 29 | TCAATCACCATGTTTTGACTCAGTTTA |
| | SEQ ID NO: 30 | AATTCCCTCAGAGAGATTATTCGATGC |
| mh02KK004 | SEQ ID NO: 31 | GATTGTTCTATGATGCTGGGTAGGGGG |
| | SEQ ID NO: 32 | TGTGTTCAGGATACCATGCCATTAG |
| mh02KK073 | SEQ ID NO: 33 | AGGAAGGCTAATGACCTCGCAAT |
| | SEQ ID NO: 34 | GACACCACCAGAACTTCTTGCTTATTA |
| mh02KK102 | SEQ ID NO: 35 | TCTCACTTATGATGCTGCTAGACTGAC |
| | SEQ ID NO: 36 | AAGAGCACATGAGATCCGCAATC |
| mh02KK105 | SEQ ID NO: 37 | GGAGCTTGCTAGAGAAGATCACGG |
| | SEQ ID NO: 38 | ATTGCTCAGCCACAAAAGATTCTCA |
| mh02KK131 | SEQ ID NO: 39 | TTTAATAGTGAAAGCAGCAAGGTTCTTC |
| | | A |
| | SEQ ID NO: 40 | TTTTCCCAGATAAATTTCAGTGTCAGCT |
| mh02KK134 | SEQ ID NO: 41 | AAAGAGTTGCATGCCGTCTGT |
| | SEQ ID NO: 42 | GTTCTAGGTGTCGTTTGCCTTAAGTTA |
| mh02KK136 | SEQ ID NO: 43 | AGTTCTCAAAGACTTCAAGACAAGTTA |
| | SEQ ID NO: 44 | TCTTTTCTCCACTTTTCAGACTTCTTGT |
| mh02KK138 | SEQ ID NO: 45 | ACCATCTCAGTGCTGAAAGAAATATAA |
| | | A |
| | SEQ ID NO: 46 | CCAGACTCATCACGTCATCCAGATA |
| mh02KK139 | SEQ ID NO: 47 | GTGACAGCTAGGTTTCATTACTGCG |
| | SEQ ID NO: 48 | AAGCCAGGATTTACCCATTTATGGAG |
| mh02KK201 | SEQ ID NO: 49 | CCAAGCTCCCTGTGATATTTCTAAA |
| | SEQ ID NO: 50 | ACTGGAAGAGTCTTTTGTTTCATAGCC |
| mh02KK202 | SEQ ID NO: 51 | GCCTTTTCCCCTTATTCTTTAAACAA |
| | SEQ ID NO: 52 | TGTTATCTCACCACTCACACATTAACTT |
| mh02KK213 | SEQ ID NO: 53 | CTCAGTAGTGAACTGCCTCAGGG |
| | SEQ ID NO: 54 | CCTTCCCCAACACTCTCTAAATATTTGC |
| mh02KK215 | SEQ ID NO: 55 | ATGCAACACTGCACCTGAGAATATG |
| | SEQ ID NO: 56 | TACCCCCTAAAAGGTTTTGAATGCAG |
| mh03KK006 | SEQ ID NO: 57 | AACCAACTAATCTACTGAAGGACTGG |
| | SEQ ID NO: 58 | CAAGAGGGACACCATATGTCAAGG |
| mh03KK007 | SEQ ID NO: 59 | CATTTTTGAAGGCTCCCATATTGCAT |
| | SEQ ID NO: 60 | AAATGTGCAGAAAGATTCCAAAGGAG |
| mh03KK008 | SEQ ID NO: 61 | AGGTACCCATCAACCTCTTTGTT |
| | SEQ ID NO: 62 | ACCTATGTGGCTGTACAATTTGTCC |
| mh03KK009 | SEQ ID NO: 63 | GAAGTCTACTCACCTCCAGGTATACC |
| | SEQ ID NO: 64 | CCAAGCCCCAAAGAGTCTGATTTTAT |
| mh03KK216 | SEQ ID NO: 65 | AAGAGCTGAAACAAGAGCATTGTGCA |
| | SEQ ID NO: 66 | CCACATTGTAACTCCTAGACCAAGAAG |
| mh04CP002 | SEQ ID NO: 67 | ACACAGAGTTTAAGGTTCCTTCCAGAA |
| | SEQ ID NO: 68 | GGGTCACTTCAGGATAATAAGCTCCT |
| mh04CP003 | SEQ ID NO: 69 | GATTTGTGTCTTCTGCATTCACAGCT |
| | SEQ ID NO: 70 | GGCTGCTCTTGTACAGCATCTC |
| mh04CP007 | SEQ ID NO: 71 | TAAATACTGTCTGCCCATGACTCCTC |
| | SEQ ID NO: 72 | AGAGCTTTGGTTTTAATGCTATTCCCT |
| mh04KK010 | SEQ ID NO: 73 | TCACTATATTTTTGAGGACACCAACCAC |
| | SEQ ID NO: 74 | TGTTGGTGCCAAGTACATCTATAAGAA |
| mh04KK011 | SEQ ID NO 75 | TTTTAAGAAAGAATAAAGAAGGACAGA |
| | | AAGCCA |
| | SEQ ID NO: 76 | GATCATGCTATCACTAAGAAAATTATGG |
| | | CAAA |
| mh04KK013 | SEQ ID NO: 77 | TGTCTAATGGCCGCTGTAGTAAA |
| | SEQ ID NO: 78 | CTTGGCAATTTAAGATGCTCAGGAATT |
| mh04KK015 | SEQ ID NO: 79 | AATTCTATCTCATCCATCTTGAGTGCAT |
| | SEQ ID NO: 80 | TATTACAGAGTGCTGCAGGTCATTC |
| mh04KK016 | SEQ ID NO: 81 | CAAAGCTAGTTTCTAAGTAAGCCATTGC |
| | SEQ ID NO: 82 | TTTTTGCCAGAGTTTTTAGTGTACTCCT |
| mh04KK017 | SEQ ID NO: 83 | ATAATGGTTGAAGGGTAGAATACACGC |
| | | A |
| | SEQ ID NO: 84 | TCGTTCAGATGAGCATGTGGTTAG |
| mh04KK019 | SEQ ID NO: 85 | TACTTGTAGCAGAGGGCCTTATC |
| | SEQ ID NO: 86 | GTTAGACAGAAGTTAGGCATGGAGTT |
| mh04KK028 | SEQ ID NO: 87 | TAATGGAAGTACTGTTTCAGTTCTGCAA |
| | SEQ ID NO: 88 | AAAAATGTTTTCCTTTTCTTCCTAGGGC |
| mh04KK029 | SEQ ID NO: 89 | CATTTACCAATGTTGGCTAATACACA |
| | SEQ ID NO: 90 | AGAACAGCATAGGAAGGCACTTAG |
| mh04KK030 | SEQ ID NO: 91 | AAATTTTGGGTCTTACCATGGTTTCAA |
| | SEQ ID NO: 92 | TTGTGTTTTTAACTGGAGGCCCTT |
| mh04KK074 | SEQ ID NO: 93 | ATATTTAAACAAAGGCTCTGGGTGTAA |
| | SEQ ID NO: 94 | CAGGGACTTCTCTAGTTTCATGTGT |
| mh05CP004 | SEQ ID NO: 95 | TGGGAACAAAGTCTCGGATGTACT |
| | SEQ ID NO: 96 | CAGCAGGACATTGACAGATACTCATTAT |
| mh05CP006 | SEQ ID NO: 97 | AGAAAAATGGCAGAGACCTTGACAC |
| | SEQ ID NO: 98 | TCTACTTTCTGTTCTCTTTGTGTTTCCG |
| mh05CP010 | SEQ ID NO: 99 | CAATCACATTGTTCCCTAGTGTCTT |
| | SEQ ID NO: 100 | AGGTGACATTGACAGAGTTGCAAATA |
| mh05KK020 | SEQ ID NO: 101 | AATAAATCGCAATGGAAGCAACAGGAA |
| | SEQ ID NO: 102 | CTCCTAGGGCTTGTGAGTCTCATA |
| mh05KK022 | SEQ ID NO: 103 | GTTGCCAATCTTACCACACCTCCA |
| | SEQ ID NO: 104 | AGCCTTTTTCTTAGGACCTGACATAG |
| mh05KK062 | SEQ ID NO: 105 | TGAACTGATCCAACTTCTCTCTCACTG |
| | SEQ ID NO: 106 | CTCAGTGCCATTGCTTATCTTCCTT |
| mh05KK078 | SEQ ID NO: 107 | CAACAAAAGAGAAAATCTGTATAGCCA |
| | | G |
| | SEQ ID NO: 108 | TTTCTGCAGTTGTTCATCTTCTACGTTA |
| mh05KK079 | SEQ ID NO: 109 | TTATTGGTCTGCTCAGAGTTTACATCAG |
| | SEQ ID NO: 110 | ACAGAACATTCTACCCAAGATTCTATGC |
| mh05KK122 | SEQ ID NO: 111 | CAACATTTTTCATGTGGCCCCTACT |
| | SEQ ID NO: 112 | GGAACAAAACAAGGTGCGGTTTT |
| mh05KK123 | SEQ ID NO: 113 | AGTGTTCTGCCAGGGTCAAAATAA |
| | SEQ ID NO: 114 | ATTGAATGCCAAAACCTCAGGGATA |
| mh05KK124 | SEQ ID NO: 115 | CAGACAAGCTGATCTGATATTTCTTTAG |
| | SEQ ID NO: 116 | GCCGCCTAAGGGATTTACCAATATG |
| mh05KK170 | SEQ ID NO: 117 | AAGACCTGAGTAGCTTCTGTTTTCTT |
| | SEQ ID NO: 118 | GGTGCTGTAATTCCCCTAAAAGCAA |
| mh06CP003 | SEQ ID NO: 119 | CAAGGAATAAAGCAGTGTGTGCCT |
| | SEQ ID NO: 120 | CCTCAAGAATCCTGGAAAATGTCAGC |
| mh06CP007 | SEQ ID NO: 121 | ACACTATTTTAAATTAGTCAACAGTTAA |
| | | GCATA |
| | SEQ ID NO: 122 | CTGAAACATCACTCAAAATAAAAGGCA |
| | | TT |
| mh06KK026 | SEQ ID NO: 123 | TCTACAACTAAGCCTTTTAACCGAGA |
| | SEQ ID NO: 124 | ATTTCACAGTTCTCTCTTGATCATGTCA |
| mh06KK030 | SEQ ID NO: 125 | GAATGCACAGAGAAATTCTTAGAGGTC |
| | | A |
| | SEQ ID NO: 126 | CTCCACCTCTTGTCTTCTAGAACCAT |
| mh06KK031 | SEQ ID NO: 127 | TCTTTGTATTCACTATTCTTGTGGCTAA |
| | SEQ ID NO: 128 | TTTCAAGATGGGATGGAGAAAGCTA |
| mh06KK080 | SEQ ID NO: 129 | CCCTATTCCAAACCTGTACCTACCT |
| | SEQ ID NO: 130 | CCCCAGTCACCCACCTAACATTTAAT |
| mh06KK101 | SEQ ID NO: 131 | GAGCCTGAGACTCTGCTACCA |
| | SEQ ID NO: 132 | GGGAGTCCCACGAGCACTG |
| mh07KK030 | SEQ ID NO: 133 | AAGTGTAGTCTGTGCAACAAGTTTCTTA |
| | SEQ ID NO: 134 | ATACAAGGATTTAGAGACCACAGCATC |
| mh07KK031 | SEQ ID NO: 135 | CTTTGGAGAAAACTGATGAGTTTAGCTT |
| | SEQ ID NO: 136 | CCTCTGTCTTCTTAACTGGCTGTAG |
| mh07KK081 | SEQ ID NO: 137 | TAAGTTGGAATCACCACCATTGACCC |
| | SEQ ID NO: 138 | ATTCATAACTCCTCCACACATCTCAGTA |
| mh07KK082 | SEQ ID NO: 139 | TGAGCTTGGAGCAGTAAAGCAGG |
| | SEQ ID NO: 140 | AGTGACATCATGTTGACTCTAACTCG |
| mh08KK032 | SEQ ID NO: 141 | AACTTGTTGCAGATTCATGGAATCATTT |
| | SEQ ID NO: 142 | AAAGAGAATAACAGTTTGACCTTGGC |
| mh09KK020 | SEQ ID NO: 143 | ATGACAGAAGAGATTTCTCTCCAGTTTG |
| | SEQ ID NO: 144 | ACTCGATTCTTTCCATTTCCATGTCG |
| mh09KK033 | SEQ ID NO: 145 | TTAAAGTCTCCTGTGTACACGGTTG |
| | SEQ ID NO: 146 | CTGTACCAATCAAGAGAAGTAGGATGG |
| | | A |
| mh09KK034 | SEQ ID NO: 147 | GATATTTGTAAGGTATTCTGGCCTAAAA |
| | | AA |
| | SEQ ID NO: 148 | AAGTATTGAAGTGATAGTTTTACAGTTT |
| | | CCT |
| mh09KK152 | SEQ ID NO: 149 | AGACTTGGAATCATTCTTCACAGGGT |
| | SEQ ID NO: 150 | GCCAGAATTAGCAGTTAGCAGTCAT |
| mh09KK153 | SEQ ID NO: 151 | TTTCTTCCTCTAAGTGGCCTCATAAATA |
| | SEQ ID NO: 152 | AGAATTAGTAAGCTCTTTCACTTGCAGT |
| mh09KK157 | SEQ ID NO: 153 | ACTAGAAGCATTAGACCAGATTACCTGC |
| | SEQ ID NO: 154 | ACAGTCCATTAGTGATGGGTTTGTT |
| mh09KK161 | SEQ ID NO: 155 | CAGAAAAACAGACTGGTCCAAAGTC |
| | SEQ ID NO: 156 | CACTGGTTTGGGAATAGAGTGCTAAG |
| mh10CP003 | SEQ ID NO: 157 | CCCCCAGAAAAGTATGTTTT |
| | SEQ ID NO: 158 | AAGACTCT |
| | | CCAAGACCAGAGAGATAACAAA |
| | | TGCAA |
| mh10KK083 | SEQ ID NO: 159 | TTTCTGAATGTGGCCTACAGTTTCAC |
| | SEQ ID NO: 160 | ATGGAATTCGAAATGATGAAGCAATGA |
| mh10KK084 | SEQ ID NO: 161 | TGTTGCTTATGCTGTTGTTCTTCACCC |
| | SEQ ID NO: 162 | GTTTGTACTTCTTTAAAGCAGGGACTG |
| mh10KK085 | SEQ ID NO: 163 | GGAGGTCAAGAAGCCTTAGTTTCTC |
| | SEQ ID NO: 164 | ATCGTGGCGCATTATCTCTTACATC |
| mh10KK086 | SEQ ID NO: 165 | GCATTCTAGCCATTGGACAATTTTGTA |
| | SEQ ID NO: 166 | TAGGTCTGCAATAATTTCCCTCTACTCA |
| mh10KK087 | SEQ ID NO: 167 | ACTGTTAAGGTCAATGACGCAGAGTA |
| | SEQ ID NO: 168 | TTACTAAAGGACTTGGTAGGTGCACATA |
| mh10KK088 | SEQ ID NO: 169 | TTTGGCCCATGGATAGAAATAAAATGTT |
| | SEQ ID NO: 170 | TTTGAAAGGCTTTTGTTATCAAGGGCTA |
| mh10KK101 | SEQ ID NO: 171 | CCATTCCCTATTCAGTGGACTCTT |
| | SEQ ID NO: 172 | AGACTCAGTGAGGTCATGACTCAA |
| mh10KK163 | SEQ ID NO: 173 | GAGCATCTTCTCCACCAGTTTGGC |
| | SEQ ID NO: 174 | TTGTCTCCTTTCAGCACAGAACC |
| mh10KK170 | SEQ ID NO: 175 | AAAGCCCACATTTTGTTAACATGACTC |
| | SEQ ID NO: 176 | ATGTAACTTCTCTGAACAGGGAAGAG |
| mh11CP003 | SEQ ID NO: 177 | AAGCAGCGATTTCCATGTTGCCC |
| | SEQ ID NO: 178 | GGCTGATTGTGGAGATGTCTCCT |
| mh11CP004 | SEQ ID NO: 179 | AGAAGCCAAAGCTCCCTAATAGCTC |
| | SEQ ID NO: 180 | GAGCCAGTTTTGTTAAAGACACAATGT |
| mh11CP005 | SEQ ID NO: 181 | TTGCTCTGAATAGTGCTTTCAGTAGTG |
| | SEQ ID NO: 182 | CAGCACTTTCTAAATAGTGATAGGCAAG |
| mh11KK036 | SEQ ID NO: 183 | CAGCTGCTTATAGTTTTGTTAAGAAG |
| | SEQ ID NO: 184 | GGACCCCTAGATAATGTCAGGATTG |
| mh11KK037 | SEQ ID NO: 185 | CTTTTGAGATCATGGAAAATTCCAGTTG |
| | SEQ ID NO: 186 | CAGAAAGAGGAACTTAAGAAGATGTGG |
| | | T |
| mh11KK038 | SEQ ID NO: 187 | GGAGTTCTAAGCAATGAGATGCTAATT |
| | SEQ ID NO: 188 | TTTCCCATAATTCCCAAAGCATGGTA |
| mh11KK039 | SEQ ID NO: 189 | AGCATCATTTCATGCTTTTGAAGTTT |
| | SEQ ID NO: 190 | ACCACCTCCTGTAACAACATCCG |
| mh11KK040 | SEQ ID NO: 191 | AGAACCCATAGGGAAACAAAGGTATGT |
| | SEQ ID NO: 192 | TTTCTCTCCTTTCAGGGAACATTACATC |
| mh11KK041 | SEQ ID NO: 193 | CATTCAGTATCTGTGTGCCTCAATGAT |
| | SEQ ID NO: 194 | CTGCAGGGTTTTCTATCCAGAACAAT |
| mh11KK089 | SEQ ID NO: 195 | CAGAATGATGAGCTGTGCAGATAGCC |
| | SEQ ID NO: 196 | GCTGTCTCTATGAACATCCCTACC |
| mh11KK090 | SEQ ID NO: 197 | TGTGATGGAGTTTATGGCCAACGG |
| | SEQ ID NO: 198 | TTATGCCCCAAATTTCACTGCTTAG |
| mh11KK091 | SEQ ID NO: 199 | AACTCCGGTCTATCCAGGTCC |
| | SEQ ID NO: 200 | TGATCCCATGGGACTACTCACG |
| mh11KK180 | SEQ ID NO: 201 | GCATCTGAGTGGCTTTCTTCTCC |
| | SEQ ID NO: 202 | CTGGGAACTTGTCCGGCTTTA |
| mh11KK187 | SEQ ID NO: 203 | TAGGAGTTTATACATGATCCTAAGGGCA |
| | SEQ ID NO: 204 | ATTTTTGGCCAAACAGAATTGTTTGC |
| mh11KK191 | SEQ ID NO: 205 | CACCAAAGGAGCTGTACCTCC |
| | SEQ ID NO: 206 | GTCAACTCCAAACAGGCTTTTTCC |
| mh12KK042 | SEQ ID NO: 207 | TTGCAAACTATGTCAAGGACACATTT |
| | SEQ ID NO: 208 | GCAAATGATCTCAGAGTTGCACAAATT |
| | | G |
| mh12KK043 | SEQ ID NO: 209 | GATGAACAGCTTGGATTGGGGC |
| | SEQ ID NO: 210 | CAGCTGAGACATAGAGAGAGGACTT |
| mh12KK045 | SEQ ID NO: 211 | AACAGGTCATGGAAGCTTTAGATCTT |
| | SEQ ID NO: 212 | AAAATCCTGGTGATAAACGTACAACCT |
| mh12KK046 | SEQ ID NO: 213 | TGTCAGCTTCTTGCGTGATAGTG |
| | SEQ ID NO: 214 | TTTTTCCCCAAGAGTCTCATCTATTAGC |
| mh12KK092 | SEQ ID NO: 215 | CATGTCTCCTTCCCTTGGTTATACC |
| | SEQ ID NO: 216 | AAAAATTGCAAGAGCAATAAGCATGTG |
| mh12KK093 | SEQ ID NO: 217 | ATCTTTTGCCTTGGCATTTGGTCTG |
| | SEQ ID NO: 218 | CTAGTTTGCTTCCTTCTATGACCCCTA |
| mh12KK202 | SEQ ID NO: 219 | GAGAGAGTGAACAGATGAGAATCAGA |
| | | AA |
| | SEQ ID NO: 220 | TTGTAATGGCCTTGGGATCAAATATTCT |
| mh13CP008 | SEQ ID NO: 221 | AGAGCTTTAGTAAGACCTCAGACTG |
| | SEQ ID NO: 222 | TAAACCAGACTGAATGTCAAAGACAAA |
| | | C |
| mh13KK047 | SEQ ID NO: 223 | GAATAACCAGTACCAGGCACGGC |
| | SEQ ID NO: 224 | TCCATCCCTTTGAGTCTATGTGTCC |
| mh13KK213 | SEQ ID NO: 225 | CTCTTGCTTCTGTCAGACACTTTTAATT |
| | SEQ ID NO: 226 | CGAGTCTCTTTTTGGTGTATTGCCA |
| mh13KK217 | SEQ ID NO: 227 | CTGGGAAACCAGCTAGAAGAAGAGA |
| | SEQ ID NO: 228 | CAAACGCACTGAGCTATTTACCTTAG |
| mh13KK218 | SEQ ID NO: 229 | GCCTCCCTTTCAGATCTTACTTAGGT |
| | SEQ ID NO: 230 | AAAATGCAACACACCTAATACTTCAGT |
| mh13KK225 | SEQ ID NO: 231 | ATGTCAGGATGCTCCACAACGGT |
| | SEQ ID NO: 232 | TCCACAGAGCATCAGCTATGAATC |
| mh13KK226 | SEQ ID NO: 233 | CTGATCTTACAAGTTCACGGCTTGT |
| | SEQ ID NO: 234 | TTCTCTATATGACCAGCCTCTTTACATG |
| mh14CP003 | SEQ ID NO: 235 | GCTGGGCATATACTCCAAAGACAG |
| | SEQ ID NO: 236 | CCAGTCTCTAGTAACTGTCCTTCTCTG |
| mh14CP004 | SEQ ID NO: 237 | GATATTAGCCCTTTGCCAGATAGATAGG |
| | | TT |
| | SEQ ID NO: 238 | GGGAAAGGATTCCCTATTTAATAAATAG |
| | | TGTC |
| mh14KK048 | SEQ ID NO: 239 | TGTCTGGAAAACTGTAGCGTGT |
| | SEQ ID NO: 240 | CCATGCACAATTAGGAACAACAGTG |
| mh14KK101 | SEQ ID NO: 241 | GATGCGGGATAAGGAATTAATCAAGGA |
| | | A |
| | SEQ ID NO: 242 | CACTATGCCTAGCTTTGTCTTGTCTTA |
| mh15CP001 | SEQ ID NO: 243 | GTACTGCAGTCACACAAAGCAGA |
| | SEQ ID NO: 244 | CTAATGAAAGGCTGCCTCTGTTCT |
| mh15CP003 | SEQ ID NO: 245 | CACACGTGCTAGTTAGGCTAAATA |
| | SEQ ID NO: 246 | CTTCCTTTGTGACTTCTGTTGCATTTAT |
| mh15CP004 | SEQ ID NO: 247 | CGCTGTGAAGTATTTAACATGCAG |
| | SEQ ID NO: 248 | GGAGGCCTTGCACTGTTTTATGA |
| mh15KK066 | SEQ ID NO: 249 | TCTATGGATCGTTCTTGCTTGTTTCT |
| | SEQ ID NO: 250 | GGGCTATTTTGTTGACTGAGAGAATG |
| mh15KK067 | SEQ ID NO: 251 | AGGGAAAATTCTTCCTTATGATGGGAAG |
| | SEQ ID NO: 252 | TCCAGTTTCAATTTTCTGCACATTGTTA |
| | | GA |
| mh15KK069 | SEQ ID NO: 253 | TATGTTGCCCAGAATTCTGAGCATAGAC |
| | SEQ ID NO: 254 | AGGGAGGAAATAATTCGCTTTGCATT |
| mh15KK095 | SEQ ID NO: 255 | CAGAATAGCACTGGATCCACAGGC |
| | SEQ ID NO: 256 | AAGCTTAATTGCCATGCCGTTTATC |
| mh16KK053 | SEQ ID NO: 257 | GTGAAGACATCGTAAAAAGATCTACCT |
| | | G |
| | SEQ ID NO: 258 | AATTTAATTGGGATCAATGCCCAAAAGG |
| mh16KK062 | SEQ ID NO: 259 | TTATTACTCTAGAGGCAGGGACTAGCCT |
| | SEQ ID NO: 260 | AGGTATCTGCTGTCAGTGTGACTAA |
| mh16KK096 | SEQ ID NO: 261 | AAGCATCTTTGGAGTTCTCTTCTCCAG |
| | SEQ ID NO: 262 | TAGACATATTCCTACATCTGTGGAATGG |
| mh16KK255 | SEQ ID NO: 263 | CTATTTCAAGGTAAGATTCTGTCTCTTA |
| | SEQ ID NO: 264 | AAGAACATATTCTAAAACAGCTGAATG |
| | | AAC |
| mh16KK302 | SEQ ID NO: 265 | AATGTCATTGACGTGATCACCTGCA |
| | SEQ ID NO: 266 | GTAGTAGGCGATGAAGAGCGT |
| mh17CP001 | SEQ ID NO: 267 | TGAGTTGAAACCCCAGTGAAACACA |
| | SEQ ID NO: 268 | CCCCAGCAATGATCTCGTAAGT |
| mh17CP006 | SEQ ID NO: 269 | AACCCTTCCTCCTAACCTCATATG |
| | SEQ ID NO: 270 | CTTACCCAACAGAACTCAAGTATTGGT |
| mh17KK014 | SEQ ID NO: 271 | TTTACTTAAAGCATAGCTTGCCTTGCC |
| | SEQ ID NO: 272 | CGGTTGCACCATTTGACATTCTATTAG |
| mh17KK052 | SEQ ID NO: 273 | AACAGGAAAGCAGATGAAACTGGC |
| | SEQ ID NO: 274 | CTACTGTGCGTGTGCGATAGC |
| mh17KK053 | SEQ ID NO: 275 | TGGATCACAACCTCACGGAGG |
| | SEQ ID NO: 276 | CGTCTTGGAAGTGAAAACACATCATA |
| mh17KK054 | SEQ ID NO: 277 | GATCGCAGCGGCTACAG |
| | SEQ ID NO: 278 | TCCATGCACAGTCCCACGA |
| mh17KK055 | SEQ ID NO: 279 | TTCATAAACAAGCAGATATGCAAGAAG |
| | | A |
| | SEQ ID NO: 280 | CATAAGCCAGTTTCCCAGTTTTCAA |
| mh17KK077 | SEQ ID NO: 281 | CTAATGCCTCTGTTCAAGCTTCTTTGC |
| | SEQ ID NO: 282 | TCAAATTCTTAGAGCTCCCAGCTGA |
| mh17KK105 | SEQ ID NO: 283 | TTTCCTTGGATTCCACACTTTGCCT |
| | SEQ ID NO: 284 | AGTAGATGGGAAATCACACGCAAAT |
| mh17KK110 | SEQ ID NO: 285 | GCCCAGTAAGAGCTTTCTTTTATGG |
| | SEQ ID NO: 286 | GATGCACGCTTATGGGTAGTGAA |
| mh17KK272 | SEQ ID NO: 287 | GTCTTCCCCCAAAACTGACAG |
| | SEQ ID NO: 288 | GGACTCTGAAGCCTCTGTACACAT |
| mh18CP003 | SEQ ID NO: 289 | CCCAAAATATTACTGCAGATGTCCTTA |
| | SEQ ID NO: 290 | AGCAGACTAATATGCCTCTGCTATTT |
| mh18CP005 | SEQ ID NO: 291 | CTCACTTTTCAGTATTCTGTTCTGAG |
| | SEQ ID NO: 292 | ATTCTGACACACAAGTTTATCCATGC |
| mh18KK285 | SEQ ID NO: 293 | TTCTCCTTGTTCTTCCCTGTGCATACC |
| | SEQ ID NO: 294 | AAGAAGCTTGAAAGTCTACAGTTGTCC |
| mh18KK293 | SEQ ID NO: 295 | CTTTCCTCCCCATCAATCACTTGGG |
| | SEQ ID NO: 296 | TCAAGGCTATGGATACCTATCTCTTCT |
| mh19CP007 | SEQ ID NO: 297 | CCCAGTTCGGCATCCGTAAGG |
| | SEQ ID NO: 298 | GGTGCCCAGATATGGAGGGAA |
| mh19KK056 | SEQ ID NO: 299 | CAACTAGAGATCACCCCATAACTCAG |
| | SEQ ID NO: 300 | TAAAAATGAAGATTCGGCCGGAC |
| mh19KK057 | SEQ ID NO: 301 | AAACAGAAGAGCATATTGGCCACAAT |
| | SEQ ID NO: 302 | GCAGTTAGGCACTAAACTATATTGTTTC |
| | | AAA |
| mh19KK299 | SEQ ID NO: 303 | CACTCCATCGTGAAAGAATAATCCTGT |
| | SEQ ID NO: 304 | GGTTAAGCTGCTTTGAGGAACAAGA |
| mh19KK301 | SEQ ID NO: 305 | GAATCCTAAGATTGTGGCTGAGAGAG |
| | SEQ ID NO: 306 | GTTCTTTCCTCCTGACATGGGAAC |
| mh20KK058 | SEQ ID NO: 307 | CCAAAAGTAAGAACTGCTTCAGGGA |
| | SEQ ID NO: 308 | ATGAGCCACATTACTTTGTTTTCTAGG |
| mh20KK059 | SEQ ID NO: 309 | TGTGGTGATGACTGAGAGATGATGC |
| | SEQ ID NO: 310 | CCATAGACCAGTGGATGCCAAC |
| mh20KK307 | SEQ ID NO: 311 | TGTGAGTCCTCTCGGTCATAGCA |
| | SEQ ID NO: 312 | CATGGCATTATCAGGGTCTGAAGAAA |
| mh21KK313 | SEQ ID NO: 313 | AAAGCTTATGTGGTAGGAGCCTAA |
| | SEQ ID NO: 314 | CAACAAGAGAGGACAAATTCTTTCACA |
| mh21KK315 | SEQ ID NO: 315 | GTACCTAGCTTAGGGTTAGACATCTG |
| | SEQ ID NO: 316 | TGTGCAGAAATAACAGAGTGAGAAAGT |
| mh21KK316 | SEQ ID NO: 317 | GAAGTCCAAAGTCAAAGTGTCAGCA |
| | SEQ ID NO: 318 | TGTTTTGGATGATATGTTTCCTTTTGTTC |
| | | ATT |
| mh21KK324 | SEQ ID NO: 319 | AGAGGAGCTTCACAAACATCCGCT |
| | SEQ ID NO: 320 | CTGCTGGTGAATCAGCAAAACCT |
| mh22KK060 | SEQ ID NO: 321 | TTATCGGCTGGAACGAGTTCA |
| | SEQ ID NO: 322 | GGTGATAACAGCTTCTCCTGTAAGG |
| mh22KK064 | SEQ ID NO: 323 | CGTGGACGCCGTGATTCAG |
| | SEQ ID NO: 324 | GTGATAGTGGGTTTTCAGTGAACG |
| mh22KK303 | SEQ ID NO: 325 | GAGCCAATCTTCAGTCAGTACCGC |
| | SEQ ID NO: 326 | CCTGTGGTCACAGTTCTTGGTC |

In an embodiment of the invention, the primer composition includes primers whose nucleotide sequences are shown in SEQ ID NO: 1 through SEQ ID NO: 326.

The invention also relates to a kit for detecting MH loci based on the next generation sequencing technology, including the primer composition, a PCR mixed solution and a PCR reaction solution.

Hereinafter, the invention is described in detail with reference to specific embodiments and accompanying drawings, so as to better understand the invention, but the following embodiments do not limit the scope of the invention.

In the following embodiments, conventional methods are use unless otherwise specified, and conventional commercially available reagents or reagents prepare according to conventional methods are used unless otherwise specified.

### Embodiment 1

The embodiment provides a method for detecting MH loci based on next generation sequencing technology using a primer composition or a kit, including step (1) through step (7) as follows.

Step (1), a sample to be tested is taken, a DNA sample is extracted, and a quantitative sample concentration is 5 ng/μL.

Step (2), a first round of multiplex PCR is conducted, a PCR amplification system and amplification conditions are shown in Table 3.

TABLE 3
The first round of PCR multiplex amplification
reaction system for library construction

| Ingredients | Volume |
|---|---|
| PCR mixed solution | 8 |
| (2*Multiplex PCR mix) | |
| PCR reaction solution | 2 |
| (2N Enhancer buffer) | |
| Primer mixed solution | 8 |
| (0.5 micromoles per liter, | |
| abbreviated as μM) | |
| DNA | 2 |

PCR reaction conditions includes: pre-denaturation at 95° C. for 15 minutes; denaturation 95° C. at for 30 seconds, annealing at 60° C. for 90 seconds, extension at 72° C. for 30 seconds, 24 cycles; heat preservation at 72° C. for 10 minutes. After reaction, a product is obtained, 1 μL of purification reaction solution is added to purify the product, and the following reactions is completed: 37° C. for 10 minutes; 50° C. for 10 minutes; 65° C. for 10 minutes, and heat preservation at 4° C. Then magnetic beads sorting is conducted.

Step (3), the purified product obtained by the step (2) is repaired make ends equal, and an adenine base (A) is added into the ends, and a reaction system thereof is shown in Table 4:

TABLE 4
The reaction system of end repair and
A adding for library construction

| Ingredients | Volume |
|---|---|
| Product obtained by the step (2) or DNA sample | 42 |
| End repair dA-tailing buffer | 6.8 |
| End repair dA-tailing enzyme | 1.2 |

PCR reaction conditions includes: 30° C. for 30 minutes; 65° C. for 30 minutes, and heat preservation at 4° C.

Step (4), ligating sequencing adapters, and a reaction system thereof is shown in Table 5:

TABLE 5
The reaction system of ligating sequencing
adapters for library construction

| Ingredients | Volume |
|---|---|
| Product obtained by the step (3) | 50 |
| Adapter mixed solution | 2.5 |
| Ligation buffer | 16 |
| Ligase | 10 |
| Nuclease-free water | 1.5 |

PCR reaction conditions includes: reaction at 25° C. for 15 minutes, and heat preservation at 4° C. Then, the reaction product is purified with purification magnetic beads to obtain a purified elution product.

Step (5), PCR amplification is conducted on the purified elution product again, and a PCR reaction system thereof is shown in Table 6:

TABLE 6
Second round of PCR reaction system for library construction

| Ingredients | Volume |
|---|---|
| Purified elution product obtained by the step (4) | 14 |
| PCR mixed solution | 25 |
| QU reagent | 3 |
| Mixed capture post-P5 primer | 4 |
| Mixed capture pre-p7 primer | 4 |

PCR reaction conditions includes: reaction at 37° C. for 15 minutes; pre-denaturation at 98° C. for 45 seconds; denaturation at 98° C. for 15 seconds, annealing at 60° C. for 30 seconds, extension at 72° C. for 30 seconds, 10 cycles; reaction at 72° C. for 5 minutes; and heat preservation at 4° C.

Step (6), purification and quantification of the library: the product obtained by the step (5) is purified again by using purification magnetic beads, and Qubit™ is used for library quantification and quality control.

Step (7), sequencing and data analysis: the constructed library is placed on MiSeq FGx™ platform for sequencing analysis to obtain sequencing data. For the obtained sequencing data, Trimmatic software is used to trim the sequencing adapters, and then BWA software is used for sequence alignment to compare the sequence with the human reference genome (hg19), and the Python tool is used to obtain MH typing.

The method can be used for individual identification and parentage testing, specifically to select 48 MH loci with good polymorphism in Asian population consisting of: mh01CP008, mh01CP012, mh01CP016, mh01KK117, mh01KK205, mh01KK211, mh02KK134, mh02KK136, mh04CP002, mh04CP003, mh04CP007, mh04KK030, mh05CP004, mh05CP006, mh05KK020, mh05KK170, mh06CP003, mh06CP007, mh09KK153, mh10CP003, mh10KK163, mh11CP003, mh11CP005, mh11KK180, mh12KK046, mh12KK202, mh13CP008, mh13KK213, mh13KK217, mh13KK218, mh13KK225, mh14CP003, mh14CP004, mh15CP001, mh15KK066, mh16KK255, mh16KK302, mh17CP001, mh17CP006, mh17KK272, mh18CP003, mh18CP005, mh19CP007, mh19KK299, mh20KK058, mh20KK307, mh21KK315, and mh21KK324. The primer sequences in Table 2 are used for detection and analysis according to the above steps.

This method can be used for ancestry inference, specifically based on MEI typing results of all 163 loci.

### Embodiment 2

The embodiment is forensic verification of the method provided in the embodiment 1. The specific experiments and results are as follows.

According to requirements of the Scientific Working Group for DNA Analysis Methods (SWGDAM), the sensitivity, accuracy, repeatability and forensic parameters of the multiplex PCR system constructed in the embodiment 1 are calculated.

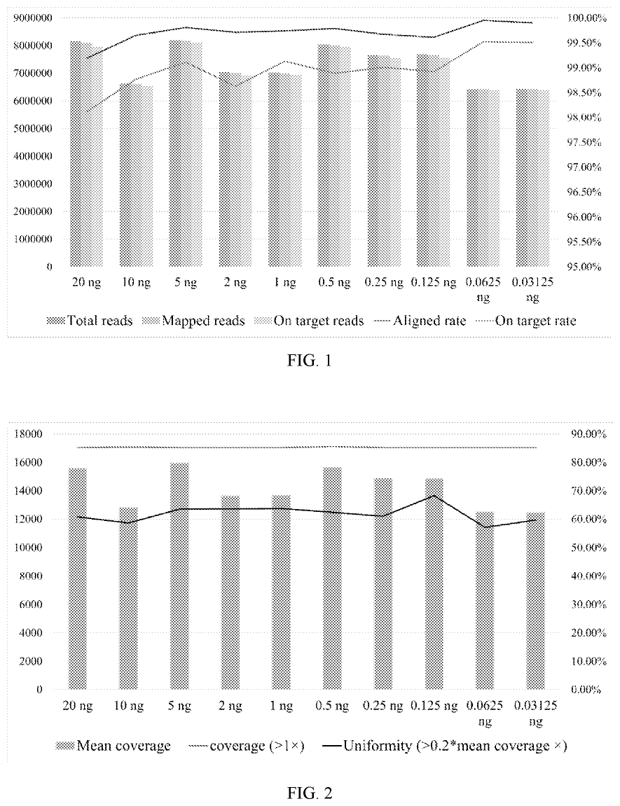

The results show that the method constructed in the embodiment 1 (for 163 MEI loci) has high sensitivity, and the complete genotyping of MEI loci can be obtained at all tested concentrations. The data statistics of next generation sequencing of DNA under different concentration gradients is shown in FIG. 1 and FIG. 2. The method has high accuracy. Sanger sequencing method is used for verification, and the results show that all SNP sites in the MEI system are consistent with the next generation sequencing results.

For 48 MEI loci with good polymorphism, the average heterozygosity of 48 loci reaches 0.7227, the polymorphism information content is greater than 0.60, the average individual identification probability reaches 0.8692, and the cumulative individual identification probability is 1-8.26×10⁻⁴⁴, the cumulative probability of exclusion in paternity of dyads and the cumulative probability of exclusion in paternity of the triad are 1-1.26×10⁻⁸ and 1-8.27×10⁻¹⁶, respectively.

### Embodiment 3

The embodiment is a comparison between the method provided in the embodiment 1 and the ForenSeg™ DNA Signature Prep Kit of the next generation sequencing platform on the analysis efficiency of mixture samples.

The autosomal STR loci in the ForenSeg™ DNA Signature Prep Kit based on the next generation sequencing platform begin to lose a large number of minor alleles below a mixture ratio of 20:1 due to its high sensitivity and stutter peaks. Samples of DNA mixtures with different mixture ratios are prepared and detected by the method provided in the embodiment 1 (for 163 MH) and ForenSeg™ DNA Signature Prep Kit, respectively, to compare the detection performance of mixtures. Table 7 shows the detection rate of the unique minor alleles in the DNA mixture samples with different mixture ratios. The results show that the detection effect of the method provided in the embodiment 1 is obviously superior to that of the STR kit of the next generation sequencing platform.

TABLE 7
Detection rate of unique minor alleles in DNA mixture samples
with different mixture ratios by the method provided in the
embodiment 1 (for 163 MH) and ForenSeq ™ DNA Signature Prep Kit

| | | Detection rate of unique minor alleles | |
|---|---|---|---|
| Mixed number | Mixture | ForenSeq ™ DNA | |
| of people | ratio | Signature Prep Ki | Embodiment 1 |
| 2 | 9:01 | 76.32% | 100.00% |
| | 19:01 | 47.37% | 94.87% |
| | 49:01:00 | 5.26% | 79.49% |
| | 99:01:00 | 2.63% | 69.23% |
| | 199:01:00 | 0.00% | 58.97% |
| 3 | 7:02:01 | 56.25% | 100.00% |
| | 17:02:01 | 56.25% | 95.16% |
| | 47:02:01 | 7.81% | 83.87% |
| | 97:02:01 | 3.13% | 75.81% |
| | 197:02:01 | 0.00% | 61.29% |
| 4 | 5:2:2:1 | 87.06% | 100.00% |
| | 13:4:2:1 | 71.76% | 98.81% |
| | 43:4:2:1 | 24.71% | 96.43% |
| | 93:4:2:1 | 8.24% | 95.24% |
| | 193:4:2:1 | 1.18% | 75.00% |
| 5 | 3:2:2:2:1 | 90.72% | 100.00% |
| | 13:2:2:1 | 76.29% | 100.00% |
| | 35:2:2:2:1 | 61.86% | 96.70% |
| | 85:8:4:2:1 | 35.05% | 91.21% |
| | 185:8:4:2:1 | 17.53% | 84.62% |

### Embodiment 4

The embodiment illustrates an application of the method provided in the embodiment 1 in the ancestry inference. The specific operation steps and results are as follows.

The MH genotyping data of 27 populations including 26 populations in the 1,000 Genomes Project and Han population of China are used to compare the genotype frequency distribution differences among 27 populations. Iₙ values of MH loci in 27 populations are calculated, the ancestry information content of the loci is evaluated, and principal component analysis is conducted.

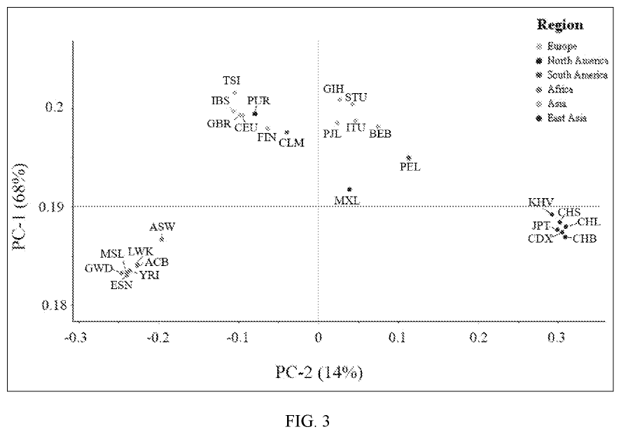

The results show that the Iₙ values of 163 MH loci are all greater than 0.185, which had high ancestry information content and could be used for ancestry inference. It can be seen from FIG. 3 that the 163 MH loci contained in the invention can clearly distinguish people in major regions around the world, and there is a clear separation among populations in Africa, East Asia, South Asia and Europe.

It can be seen from the above embodiments that the primer composition, the kit and the method provided by the invention provide a new detection means for individual identification, parentage testing, mixture analysis, ancestry inference, etc. in the field of forensic medicine.

The specific embodiments of the invention are described in detail above, by way of examples only, and the invention is not limited to the specific embodiments described above. For those skilled in the art, any equivalent modifications and substitutions of the invention are also included in the scope of the invention. Therefore, the equivalent changes and modifications made without departing from the spirit and scope of the invention should be included within the scope of the invention.

What is claimed is:

1. A primer composition for detecting microhaplotype (MH) loci based on next generation sequencing technology, comprising: one or more pairs of amplification primers of 163 MH loci;
wherein the 163 MH loci consist of: mh01CP007, mh01CP008, mh01CP012, mh01CP016, mh01KK001, mh01KK070, mh01KK072, mh01KK106, mh01KK117, mh01KK172, mh01KK205, mh01KK210, mh01KK211, mh02CP004, mh02KK003, mh02KK004, mh02KK073, mh02KK102, mh02KK105, mh02KK131, mh02KK134, mh02KK136, mh02KK138, mh02KK139, mh02KK201, mh02KK202, mh02KK213, mh02KK215, mh03KK006, mh03KK007, mh03KK008, mh03KK009, mh03KK216, mh04CP002, mh04CP003, mh04CP007, mh04KK010, mh04KK011, mh04KK013, mh04KK015, mh04KK016, mh04KK017, mh04KK019, mh04KK028, mh04KK029, mh04KK030, mh04KK074, mh05CP004, mh05CP006, mh05CP010, mh05KK020, mh05KK022, mh05KK062, mh05KK078, mh05KK079, mh05KK122, mh05KK123, mh05KK124, mh05KK170, mh06CP003, mh06CP007, mh06KK026, mh06KK030, mh06KK031, mh06KK080, mh06KK101, mh07KK030, mh07KK031, mh07KK081, mh07KK082, mh08KK032, mh09KK020, mh09KK033, mh09KK034, mh09KK152, mh09KK153, mh09KK157, mh09KK161, mh10CP003, mh10KK083, mh10KK084, mh10KK085, mh10KK086, mh10KK087, mh10KK088, mh10KK101, mh10KK163, mh10KK170, mh11CP003, mh11CP004, mh11CP005, mh11KK036, mh11KK037, mh11KK038, mh11KK039, mh11KK040, mh11KK041, mh11KK089, mh11KK090, mh11KK091, mh11KK180, mh11KK187, mh11KK191, mh12KK042, mh12KK043, mh12KK045, mh12KK046, mh12KK092, mh12KK093, mh12KK202, mh13CP008, mh13KK047, mh13KK213, mh13KK217, mh13KK218, mh13KK225, mh13KK226, mh14CP003, mh14CP004, mh14KK048, mh14KK101, mh15CP001, mh15CP003, mh15CP004, mh15KK066, mh15KK067, mh15KK069, mh15KK095, mh16KK053, mh16KK062, mh16KK096, mh16KK255, mh16KK302, mh17CP001, mh17CP006, mh17KK014, mh17KK052, mh17KK053, mh17KK054, mh17KK055, mh17KK077, mh17KK105, mh17KK110, mh17KK272, mh18CP003, mh18CP005, mh18KK285, mh18KK293, mh19CP007, mh19KK056, mh19KK057, mh19KK299, mh19KK301, mh20KK058, mh20KK059, mh20KK307, mh21KK313, mh21KK315, mh21KK316, mh21KK324, mh22KK060, mh22KK064, and mh22KK303.

2. The primer composition according to claim 1, specifically comprising one or more pairs of the amplification primers with nucleotide sequences shown in SEQ ID NO: 1 through SEQ ID NO: 326.

3. The primer composition according to claim 2, specifically comprising the amplification primers with the nucleotide sequences shown in SEQ ID NO: 1 through SEQ ID NO: 326.

4. A kit for detecting MR loci based on next generation sequencing technology, comprising the primer composition according to claim 1, a polymerase chain reaction (PCR) mixed solution, and a PCR reaction solution.

5. The kit according to claim 4, wherein the kit is used for individual identification, parentage testing, mixture analysis and ancestry inference;
wherein the individual identification and the parentage testing are determined based on typing results of 48 MR loci, and the 48 MR loci consist of: mh01CP008, mh01CP012, mh01CP016, mh01KK117, mh01KK205, mh01KK211, mh02KK134, mh02KK136, mh04CP002, mh04CP003, mh04CP007, mh04KK030, mh05CP004, mh05CP006, mh05KK020, mh05KK170, mh06CP003, mh06CP007, mh09KK153, mh10CP003, mh10KK163, mh11CP003, mh11CP005, mh11KK180, mh12KK046, mh12KK202, mh13CP008, mh13KK213, mh13KK217, mh13KK218, mh13KK225, mh14CP003, mh14CP004, mh15CP001, mh15KK066, mh16KK255, mh16KK302, mh17CP001, mh17CP006, mh17KK272, mh18CP003, mh18CP005, mh19CP007, mh19KK299, mh20KK058, mh20KK307, mh21KK315, and mh21KK324.

6. A method for detecting MR loci based on next generation sequencing technology using the kit according to claim 4, comprising:
step 1, taking a sample to be tested, extracting a DNA sample from the sample to be tested, and quantifying the DNA sample;
step 2, preparing a multiplex PCR system, and conducting a first round of multiplex PCR; obtaining a product after the first round of multiplex PCR is completed, adding a purification reaction solution to purify the product, and conducting magnetic bead sorting on the purified product;
step 3, repairing the purified product to make ends equal and adding an adenine base (A) into the ends, then ligating sequencing adapters on the ends to obtain a complemented product, and then purifying the complemented product again using purification magnetic beads to obtain a purified elution product;
step 4, conducting a PCR reaction on the purified elution product using a reaction system to obtain a reaction product for constructing a library, wherein the reaction system comprises the purified elution product, a PCR mixed solution, a QU reagent, a mixed post-P5 primer, and a mixed pre-p7 primer;
step 5, conducting purification and quantification on the library, specifically comprising: purifying the reaction product by using purification magnetic beads, and conducting quantification and quality control on the library by using Qubit™; and
step 6, conducting sequencing and data analysis, specifically comprising: using the constructed library on a MiSeq FGx™ platform for sequencing to obtain sequencing data; trimming the sequencing adapters of the obtained sequencing data by using a Trimmatic software to obtain a sequencing sequence, then comparing the sequencing sequence with human reference genome hg19 by using a burrows-wheeler aligner (BWA) software, and obtaining MEI typing by using a Python tool.

7. The method according to claim 6, wherein a concentration of the DNA sample is 5 nanograms per microliter (ng/μL).

8. The method according to claim 6, wherein the multiplex PCR system comprises 20 μL total reaction volume, specifically comprising 8 μL of the PCR mixed solution, 2 μL of the PCR reaction solution, 8 μL of primer mixed solution, and 2 μL of the DNA sample; and reaction conditions of the multiplex PCR in the step 2 comprises: pre-denaturation at 95° C. for 15 minutes; denaturation at 95° C. for 30 seconds, annealing at 60° C. for 90 seconds, extension at 72° C. for 30 seconds, 24 cycles, and heat preservation at 72° C. for 10 minutes.

9. The method according to claim 6, wherein a reaction system of the repairing the purified product to make ends equal and adding A into the ends in the step 3 comprises 50 μL total reaction volume, specifically comprising 42 μL of the purified product in the step 2, 6.8 μL of end repair dA-tailing buffer, and 1.2 μL of end repair dA-tailing enzyme; and reaction conditions of the repairing the purified product to make ends equal and adding A into the ends in the step 3 comprises: reaction at 30° C. for 30 minutes;
then reaction at 65° C. for 30 minutes; and finally heat preservation at 4° C.

10. The method according to claim 6, wherein a reaction system of the ligating sequencing adapters on the ends in the step 3 comprises 80 μL total reaction volume, specifically comprising 50 μL of the purified elution product in the step 3, 2.5 μL of adapter mixed solution, 16 μL of ligation buffer, 10 μL of ligase, and 1.5 μL of nuclease-free water; and reaction conditions of the ligating sequencing adapters in the step 3 comprises: reaction at 25° C. for 15 minutes, and heat preservation at 4° C.

11. The method according to claim 6, wherein a reaction system of the PCR reaction of the step 4 comprises 50 μL total reaction volume, specifically comprising 14 μL of the elution purified product of the step 3, 25 μL of the PCR mixed solution, 3 μL of the QU reagent, 5 μL of the mixed capture post-P5 primer, and 5 μL of the mixed capture pre-p7 primer; and reaction conditions of the PCR reaction in the step 4 comprises: reaction at 37° C. for 15 minutes; pre-denaturation at 98° C. for 45 seconds; denaturation at 98° C. for 15 seconds, annealing at 60° C. for 30 seconds, extension at 72° C. for 30 seconds, 10 cycles, then reaction at 72° C. for 5 minutes, and heat preservation at 4° C.
